(19) United States
(12) Patent Application Publication
(10) Pub. No.: US 2009/0170917 A1
(43) Pub. Date: Jul. 2, 2009

(54) Acid Mimic Compounds for the Inhibition of Isoprenyl-S-Cysteinyl Methyltransferase

(76) Inventors: Seung-Yub Lee, Palisades Park, NJ (US); Eduardo Perez, Somerset, NJ (US); Keshava Rapole, Edison, NJ (US); Gopal Sarngadharan, Robbinsville, NJ (US); Gregory B. Stock, Skillman, NJ (US); Jeffry B. Stock, Princeton, NJ (US); Maxwell Stock, Rocky Hill, NJ (US); Michael Voronkov, Pennington, NJ (US); Peter Wolanin, Princeton, NJ (US)

Correspondence Address: CHOATE, HALL & STEWART LLP, TWO INTERNATIONAL PLACE, BOSTON, MA, 02110, US

(21) Appl. No.: 12/326,746

(22) Filed: Dec. 2, 2008

Related U.S. Application Data
(60) Provisional application No. 61/005,129, filed on Dec. 3, 2007.
(60) Provisional application No. 61/005,866, filed on Dec. 7, 2007.
(60) Provisional application No. 61/007,234, filed on Dec. 10, 2007.

(51) Int. Cl. A61K 31/41; A61K 31/27; C07C 271/22; A61K 31/16; C07C 243/34; C07D 257/04; A61P 11/00

(52) U.S. Cl. 514/381; 514/478; 560/160; 564/151; 514/614; 548/253

(57) ABSTRACT

Among other things, the present invention provides novel compounds capable of effectively inhibiting inflammatory responses that are mediated by G-proteins or GPCRs in neutrophils, macrophages and platelets. In particular, compounds of the present invention act as inhibitors of edema, inhibitors of erythema and inhibitors of MPO (myeloperoxidase), pharmaceutical compositions containing the same compounds and the use thereof for the treatment of diseases that may benefit from edema, erythema and MPO inhibition, such as inflammation (acute or chronic), asthma, autoimmune diseases, and chronic obstructive pulmonary disease (COPD) (e.g., emphysema, chronic bronchitis and small airways disease, etc.), inflammatory responses of the immune system, skin diseases (e.g., reducing acute skin irritation for patients suffering from rosacea, atopic dermatitis, seborrheic dermatitis, psoriasis), irritable bowel syndrome (e.g., Chron's disease and ulcerative colitis, etc.), and central nervous system disorders (e.g., Parkinson's disease).

### RELATED APPLICATIONS

This application claims priority to U.S. provisional patent application Ser. No. 61/005,129, filed Dec. 3, 2007, U.S. provisional patent application Ser. No. 61/005,866, filed Dec. 7, 2007, and U.S. provisional patent application Ser. No. 61/007,234, filed Dec. 10, 2007, the entire disclosure of each of which is incorporated herein by reference.

### BACKGROUND

Inflammation often is a bodily response to infection or injury in which cells involved in detoxification and repair are mobilized to the compromised site by inflammatory mediators. The infection or injury can be a result of acute or chronic disease, disorders, conditions or trauma, environmental conditions, or aging. Examples include diseases, disorders, syndromes, conditions and injuries of the cardiovascular, digestive, integumentary, muscular, nervous, reproductive, respiratory and urinary systems, as well as, diseases, disorders, syndromes, conditions and injuries of tissue and cartilage such as atherosclerosis, irritable bowel syndrome, psoriasis, tendonitis, Alzheimer's disease and vascular dementia, multiple sclerosis, diabetes, endometriosis, asthma and kidney failure.

N-acetyl-5-farnesyl-L-cysteine (“AFC”), also referred to as N-acetyl-5-trans, trans-farnesyl-L-cysteine, is a signal transduction modulator that has been shown to reduce inflammation in mice. AFC is a polyisoprenyl-protein inhibitor, and has been shown to be a competitive inhibitor of membrane-associated isoprenyl-5-cysteinyl methyltransferase. AFC has also been shown to block some neutrophil, macrophage, and platelet responses in vitro. Treatment of inflammatory diseases or disorders with traditional anti-inflammatory drugs, e.g., corticosteroids and non-steroidal anti-inflammatory drugs (“NSAIDS”) can cause multiple side effects, e.g., appetite and weight gain, excess sweating, high blood pressure, nausea, vomiting, diarrhea, etc.

Inflammation often is characterized by a strong infiltration of polymorphonuclear leukocytes at the site of inflammation, particularly neutrophils. These cells promote tissue damage by releasing toxic substances at the vascular wall or in uninjured tissue. Neutrophil infiltration results from amplifying cascades of cell-cell communication involving signal transduction proteins, such as G-proteins, that can facilitate intracellular regulation and intercellular communication by interacting with a wide range of different regulatory receptor-transducer proteins, such as membrane bound receptors. For these interactions to occur, many of the signal transduction proteins, including virtually all G-proteins, first must be modified by the post-translational addition of a C₁₅ farnesyl or a C₂₀ geranylgeranyl polyisoprenoid moiety in thioether linkage to a cysteine residue located at or near the carboxyl terminus within a so-called CAAX box or related cysteine-containing sequence.

Carboxy-terminal polyisoprenoid cysteines that ultimately result from these modifications may be subject to methylesterification by a specific membrane associated S-adenosylmethionine-dependent soprenyl-S-cysteinyl methyltransferase. Compounds that can inhibit these enzymatic reactions or otherwise alter the interactions among polyisoprenylated signal transduction proteins, such as G-proteins and the protein regulatory targets with which they interact, or other intracellular signaling proteins, may be used to mitigate leukocyte responses and, theoretically, to treat inflammatory-related conditions. (See e.g., Volker, et al., Methods Enzymol, 1995, 250: 216-225).

One signal transduction modulator compound is N-acetyl-5-farnesyl-L-cysteine (“AFC”), also referred to as N-acetyl-5-trans, trans-farnesyl-L-cysteine. AFC has been shown to be a competitive inhibitor of membrane-associated isoprenyl-5-cysteinyl methyltransferase and to block some neutrophil, macrophage, and platelet responses in vitro. Laboratory results also indicate that AFC effectively reduces dermal inflammation in mice. AFC requires high concentrations for efficacy, which would seem to preclude its use in vivo.

Numerous drugs have been used to treat inflammation, all of which suffer from some side effects, some of which are serious. For example, common side effects of corticosteroids include increased appetite and weight gain, deposits of fat, in chest, face, upper back and stomach, water and salt retention leading to swelling and edema, high blood pressure, diabetes, excess sweating, telangiectasia (dilation of capillaries), slowed healing of wounds, osteoporosis, cataracts, acne, hirsutism, muscle weakness, atrophy of the skin and mucous membranes, an increased susceptibility to infection, and stomach ulcers.

In another example, studies have demonstrated an increased risk of cardiovascular events associated with the use of the Cox-II inhibitors, such as such as Celebrex® and Vioxx® (See, e.g., Solomon et al., N. Engl. J. Med 2005; 352: 1071-80; Nussmeier et al, N. Engl. J. Med. 2005; 352: 1081-91).

Side-effects of NSAIDS such as aspirin and ibuprofen vary between drugs, but generally include nausea, vomiting, diarrhea, constipation, decreased appetite, rash, dizziness, headache, drowsiness and photosensitivity. NSAIDs also may cause fluid retention, leading to edema. The most serious side effects of NSAIDs use are kidney failure, liver failure, ulcers and prolonged bleeding after an injury or surgery. NSAIDs can produce shortness of breath in individuals allergic to them. People with asthma are at a higher risk for experiencing serious allergic reaction to NSAIDS. Individuals with a serious allergy to one NSAID are likely to experience a similar reaction to a different NSAID.

Thus, there is a need for a non-steroidal anti-inflammatory compound that lacks the side effects of corticosteroids and NSAIDS. It has been found that signal transduction modulator compounds may impede inflammation. Without being bound by any particular theory, the impediment of inflammation may be a result of the ability of signal transduction modulator compounds to alter cell to cell signaling. The present invention, therefore, is directed to a novel signal transduction modulator compound for treating and preventing inflammation, and for other unmet needs.

Other background and methods may be found in U.S. Pat. Nos. 5,043,268, 5,202,456, and 5,705,528, as well as US patent applications 2005/0277694 and 2007/0004803 each of which are incorporated herein by reference.

### SUMMARY

Among other things, the present invention provides novel compounds that modulate the G-protein signaling cascade. The present invention provides certain compounds that are structurally related to N-acetyl-5-farnesyl-L-cysteine (“AFC”).

The present invention demonstrates desirable characteristics of certain such compounds. For example, among other things, the present invention demonstrates that certain such compounds and/or compositions show inhibition of edema, erythema and dermal neutrophil infiltration, as measured by inhibition of MPO (myeloperoxidase).

In certain embodiments, compounds provided by the present invention have the structure set forth in formula I,

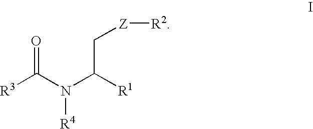

In certain embodiments, compounds provided by the present invention have the structure set forth in formula Ia,

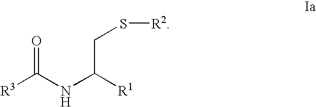

In certain embodiments, compounds of formulae I and/or Ia are provided in a pharmaceutically acceptable salt form. Other embodiments are described in more detail below.

The present invention also provides compositions containing compounds described herein, methods of preparing such compounds and/or compositions, and methods of using such compounds and/or compositions.

In certain embodiments, the present invention provides uses of provided compounds and/or compositions in the treatment of inflammation and/or misregulation of cellular processes. In certain embodiments, the present invention provides uses of provided compounds and/or compositions in the treatment of diseases that may benefit from edema inhibition, erythema inhibition and/or MPO inhibition, such as treating or lessening the severity of inflammatory diseases or disorders selected from inflammation (acute or chronic), asthma, autoimmune diseases, and chronic obstructive pulmonary disease (COPD) (e.g., emphysema, chronic bronchitis and small airways disease, etc.), inflammatory responses of the immune system, skin diseases (e.g., reducing acute skin irritation for patients suffering from rosacea, atopic dermatitis, seborrheic dermatitis, psoriasis), irritable bowel syndrome (e.g., Chron's disease and ulcerative colitis, etc.), and central nervous system disorders (e.g., Parkinson's disease).

While various aspects of the disclosure herein are illustrated through the use of certain compounds, it is an object of the present invention to extend the various embodiments described herein utilizing a compound or a composition comprising a compound of formula I. Various other compounds and/or compositions as described herein would be known to those skilled in the art made aware of this disclosure. As described more fully below, in the accompanying figures, examples and descriptions, a related object of this disclosure includes the provision of various compounds and/or compositions that can be used in any of a variety of applications.

### DEFINITIONS

“Acyl group”: As used herein, the term “acyl group” includes a group —R—C(═O)—, where R is an organic group, for example but not limited to, an alkyl group. An example may be the acetyl group —CH₃—C(═O)—, referred to herein as “Ac”.

“Aliphatic group”: As used herein, the term “aliphatic group” means a hydrocarbon group, but not limited to, straight or branched chain hydrocarbon chains, such as straight or branched chain alkanes, straight or branched chain alkenes with one or more double bonds, and straight or branched chain alkynes with one or more triple bonds and optionally also with one or more double bonds, for example. An aliphatic group may optionally be substituted with one or more suitable substituents. The term “aliphatic” is used interchangeably herein.

An aliphatic group is a straight or branched chain alkyl group, with about 10 to about 25 carbon atoms or a straight or branched chain alkenyl group, with about 10 to about 25 carbon atoms and one or more double bonds. In one embodiment, preferred aliphatic groups may include all stereoisomers and double bond isomers of farnesyl or geranylgeranyl, unsubstituted or substituted with one or more suitable substituents.

“Alkenyl group”: As used herein, the term “alkenyl group” means a monovalent, unbranched or branched hydrocarbon chain having one or more double bonds therein. The double bond of an alkenyl group can be unconjugated or conjugated to another unsaturated group. Suitable alkenyl groups may include, but are not limited to (C₂-C₆) alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl group may be unsubstituted or optionally substituted with one or two suitable substituents. The term “alkenyl” is used interchangeably herein.

“Alkoxy group”: As used herein, the term “alkoxy group” means an —O-alkyl group, where alkyl is as defined above. An alkoxy group may be unsubstituted or optionally substituted with one or more suitable substituents. The term “alkoxy” is used interchangeably herein.

“Alkoxycarbony group”: As used herein, the term “alkoxycarbonyl group” means a monovalent group of the formula —C(═O)—O-alkyl. Preferably, the alkyl group of an alkoxycarbonyl group is from 1 to 8 carbon atoms in length, referred to herein as a “lower alkoxycarbonyl group.”

“Alkyl group”: As used herein, the term “alkyl group” means a saturated, monovalent, unbranched or branched hydrocarbon chain. Examples of alkyl groups include, but are not limited to, (C₁-C₆) alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl group can be unsubstituted or optionally substituted with one or two suitable substituents. The term “alkyl” is used interchangeably herein.

“Alkynyl group”: As used herein, the term “alkynyl group” means monovalent, unbranched or branched hydrocarbon chain having one or more triple bonds therein. The triple bond of an alkynyl group may be unconjugated or conjugated to another unsaturated group. Suitable alkynyl groups may include, but are not limited to, —(C₂-C₆)alkynyl groups, such as ethynyl, propynyl, butynyl, pentynyl, hexynyl, methylpropynyl, 4-methyl-1-butynyl, 4-propyl-2-pentynyl, and 4-butyl-2-hexynyl. An alkynyl group may be unsubstituted or optionally substituted with one or two suitable substituents. The term “alkynyl” is used interchangeably herein.

“Amide”: An “amide” includes compounds that have a trivalent nitrogen attached to a carbonyl group —(C(═O)—NH₂), such as for example methylamide, ethylamide, propylamide, and the like.

“Animal”: The term animal, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). A non-human animal may be a transgenic animal.

“Aryl group”: As used herein, the term “aryl group” means a monocyclic or polycyclic-aromatic radical having carbon and hydrogen atoms. Examples of suitable aryl groups may include, but are not limited to, phenyl, tolyl, anthacenyl, fluorenyl, indenyl, azulenyl, naphthyl, 1-naphthyl, 2-naphthyl, and biphenyl as well as benzo-fused carbocyclic moieties such as 5, 6, 7, 8-tetrahydronaphthyl. An aryl group can be unsubstituted or optionally substituted with one or two suitable substituents as defined below. An aryl group optionally may be fused to a cycloalkyl group, fused to another aryl group, fused to a heteroaryl group, or fused to a heterocycloalkyl group. Preferred aryl groups may include, but are not limited to, monocyclic or bicyclic aromatic hydrocarbon radicals of 6 to 12 ring atoms, and optionally substituted independently with one or more suitable substituents. The term “aryl” is used interchangeably herein.

“Aryloxy group”: As used herein, the term “aryloxy group” means an —O-aryl group, wherein aryl is as defined above. An aryloxy group may be unsubstituted or optionally substituted with one or more suitable substituents. The term “aryloxy” is used interchangeably herein.

“Associated with”: When two entities are “associated with” one another as described herein, they are linked by a direct or indirect covalent or non-covalent interaction. Preferably, the association is covalent. Desirable non-covalent interactions include hydrogen bonding, van der Waals interactions, hydrophobic interactions, magnetic interactions, electrostatic interactions, etc.

“Carbamoyl”: As used herein, the term “carbamoyl” group means the radical —C(═O)N(R¹)₂, where R¹ is chosen from the group consisting of hydrogen, alkyl, and aryl.

“Carbonyl”: As used herein, a “carbonyl” group is a divalent group of the formula —C(═O).

“Composition”: The term “composition”, as in pharmaceutical composition, encompasses a product with active ingredient(s) and a carrier with one or more inert ingredient(s). Accordingly, pharmaceutical compositions may encompass a composition with a compound and a pharmaceutically acceptable carrier.

“Cyclic radical”: As used herein, the term “cyclic radical” means an aryl group, a cycloalkyl group, a heterocycloalkyl group or a heteroaryl group.

“Cycloalkyl group”: As used herein, the term “cycloalkyl group” means a monocyclic or polycyclic saturated ring with carbon and hydrogen atoms and having no carbon—carbon multiple bonds. Examples of cycloalkyl groups may include, but are not limited to, (C₃-C₇)cycloalkyl groups, such as cyclopropyl, cyclobutyl, cyclopentyl, cyclohexyl, and cycloheptyl, and saturated cyclic and bicyclic terpenes. A cycloalkyl group may be unsubstituted or optionally substituted with one or two suitable substituents as defined below. A cycloalkyl group optionally may be fused to another cycloalkyl group, fused to an aryl group, fused to a heteroaryl group, or fused to a heterocycloalkyl group. The term “cycloalkyl” is used interchangeably herein.

“G-protein mediated condition”: The term “G-protein mediated condition”, as used herein means any disease or other deleterious condition for which the appearance, incidence, and/or severity of one or more symptoms correlates with changes in a G-protein signaling cascade. In some embodiments, one or more symptoms of the disease or condition is caused by a defect or alteration in G-protein signaling.

“Halogen”: As used herein, the term “halogen” means fluorine, chlorine, bromine, or iodine. Correspondingly, the meaning of the terms “halo” and “Hal” encompass fluoro, chloro, bromo, and iodo.

“Heteroaryl group”: As used herein, the term “heteroaryl group” means a monocyclic- or polycyclic aromatic ring comprising carbon atoms, hydrogen atoms, and one or more heteroatoms, preferably 1 to 4 heteroatoms, independently selected from nitrogen, oxygen, and sulfur groups may include, but are not limited to, pyrazolyl, triazolyl, tetrazolyl, pyridyl, pyridazinyl, pyrazyl, indolyl, triazinyl, pyrrolyl, pyrazolyl, imidazolyl, (1,2,3)-triazolyl, (1,2,4)-riazolyl, pyrazinyl, pyrimidinyl, tetrazolyl, furyl, thienyl, 4H-1,4-thiazine, isoxazolyl, thiazolyl, phienyl, isoxazolyl, oxazolyl, pyrazolyl, tetrazolyl, triazolyl, oxadiazolyl, thiadiazolyl, isoxazolyl, triazinyl, and pyrazinyl. Bicyclic heteroaromatic rings may include, but are not limited to, benzothiadiazolyl, indolyl, benzothiophenyl, benzofuryl, benzimidazolyl, purinyl, benzisoxazolyl, benzothiazolyl, quinolinyl, benzotriazolyl, benzoxazolyl, isoquinolinyl, purinyl, furopyridinyl and thienopyridinyl. A heteroaryl can be unsubstituted or optionally substituted with one or more suitable substituents as defined below. A heteroaryl group optionally may be fused to another heteroaryl group, fused to an aryl group, fused to a cycloalkyl group, or fused to a heterocycloalkyl group. The term “heteroaryl” is used interchangeably herein.

“Heterocyclic radical” or “heterocyclic ring”: As used herein, the terms “heterocyclic radical” or “heterocyclic ring” mean a heterocycloalkyl group or a heteroaryl group.

“Heterocycloalkyl group”: As used herein, the term “heterocycloalkyl group” means a monocyclic or polycyclic ring with carbon and hydrogen atoms and at least one heteroatom, preferably, 1 to 3 heteroatoms selected from nitrogen, oxygen, and sulfur. A heterocycloalkyl group may be fused to an aryl or heteroaryl group. Examples of heterocycloalkyl groups may include, but are not limited to, pyrrolidinyl, pyrrolidino, piperidinyl, piperidino, piperazinyl, piperazino, morpholinyl, morpholino, thiomorpholinyl, thiomorpholino, and pyranyl. A heterocycloalkyl group may be unsubstituted or optionally substituted with one or more suitable substituents as defined below. A heterocycloalkyl group optionally may be fused to a cycloalkyl group, fused to an aryl group, fused to a heteroaryl group, or fused to another heterocycloalkyl group. The term “heterocycloalkyl” is used interchangeably herein.

“In combination”: As used herein, the phrase “in combination” refers to agents that are simultaneously administered to a subject. It will be appreciated that two or more agents are considered to be administered “in combination” whenever a subject is simultaneously exposed to both (or more) of the agents. Each of the two or more agents may be administered according to a different schedule; it is not required that individual doses of different agents be administered at the same time, or in the same composition. Rather, so long as both (or more) agents remain in the subject's body, they are considered to be administered “in combination”.

“Independently selected”: The term “independently selected” is used herein to indicate that the R groups can be identical or different.

“Modulate”: The term “modulate” refers to change in a parameter (e.g., a change in a binding interaction or an activity, etc.). Modulation can refer to an increase or a decrease in the parameter (e.g., an increase or decrease in binding, an increase or decrease in activity, etc.).

“Modulator”: The term “modulator” refers to an agent that alters level and/or activity of its target (e.g., in the GPCR signal transduction pathway). In some embodiments, a modulator alters interaction between a protein in the GPCR signal transduction pathway and one or more other entities. In some embodiments, a modulator alters interaction between a modulator alters interaction between a protein in the GPCR signal transduction pathway and a substrate. Determination of whether an agent is a modulator can be performed directly or indirectly. Determination of whether an agent modulates an interaction can be performed directly, e.g., using an assay that detects the interaction between a protein in the GPCR signal transduction pathway and a substrate. Determination of whether an agent modulates an interaction can be performed with a technique that indirectly detects modulation, e.g., a technique that detects a biological activity that is downstream of, and dependent on, the protein-substrate interaction.

“Oxo group”: As used herein, an “oxo group” is a group of the formula (═O).

“Pharmaceutically acceptable ester”: The term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic, and alkanedioic acids, in which each alkyl or alkenyl moiety advantageously has not more than 6 carbon atoms. Examples of particular esters include formates, acetates, propionates, butyrates, acrylates, and ethylsuccinates. In certain embodiments, the esters are cleaved by enzymes such as esterases.

“Pharmaceutically acceptable prodrugs”: The term “pharmaceutically acceptable prodrugs” as used herein refers to those prodrugs of compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

“Pharmaceutically acceptable salt(s)”: The term “pharmaceutically acceptable salt(s)”, as used herein may include but is not limited to salts of acidic or basic groups that may be present in compounds of the present invention. Compounds that are basic in nature may be capable of forming a wide variety of salts with various inorganic and organic acids. Such non-toxic salts, i.e., salts containing pharmacologically acceptable anions, may include but are not limited to hydrochloride, hydrobromide, hydroiodide, nitrate, sulfate, bisulfate, phosphate, acid phosphate, isonicotinate, acetate, lactate, salicylate, citrate, acid citrate, tartrate, oxalate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, ptoluenesulfonate and pamoate (i.e., 1,1′-methylene-bis-(2-hydroxy-3-naphthoate)) salts. Compounds of the present invention that may include an amino moiety also may form pharmaceutically acceptable salts with various amino acids, in addition to the acids mentioned above. Compounds that may be acidic in nature are capable of forming base salts with various pharmacologically acceptable cations. Examples of such salts may include alkali metal or alkaline earth metal salts and, particularly, calcium, magnesium, sodium lithium, zinc, potassium, and iron salts. Other such salts may include pharmaceutically acceptable organic bases such as ammonia, arginine, benethamine, benzathine, deanol, diethanolamine, diethylamine, -2-diethylaminoethanol, ethanolamine, ethylenediamine, lysine, -2-hydroxyethylmorpholine, piperazine, -2-hydroxyethylpyrrolidine, triethanolamine, tromethamine.

“Prophylactically effective”, “preventing” or “preventive”: As used herein, the term “prophylactically effective” or “preventive” means the amount of a compound that will prevent or inhibit affliction a medical condition that a medical doctor or other clinician is trying to prevent, inhibit before a patient begins to suffer from the specified disease or disorder.

“Skin Irritant”: As applied to skin or a skin equivalents, elicits a cellular response characterized by the used herein, the term “skin irritant” refers to a compound that, when expression of an “irritant responsive gene.” Examples of known skin irritants include, but are not limited to, sodium dodecyl sulfate (“SDS”), calcipotriol, and trans-retinoic acid. The term “skin irritant” is also intended to encompass unknown or suspected irritants, including but not limited to, those containing in some pharmaceuticals, cosmetics, and consumer products.

“Small Molecule”: As used herein, the term “small molecule” refers to an organic compound either synthesized in the laboratory or found in nature. Typically, a small molecule is characterized in that it contains several carbon-carbon bonds, and has a molecular weight of less than 1500, although this characterization is not intended to be limiting for the purposes of the present invention. Examples of “small molecules” that occur in nature include, but are not limited to, taxol, dynemicin, and rapamycin. Examples of “small molecules” that are synthesized in the laboratory include, but are not limited to, the inventive compounds incorporated herein.

“Suitable substituent”: As used herein, the term “suitable substituent” means a group that does not nullify the therapeutic or pharmaceutical utility of compounds of the present invention or the synthetic utility of the intermediates useful for preparing them. Examples of suitable substituents may include, but are not limited to: alkyl; alkenyl; alkynyl; aryl; heteroaryl; heterocycloalkyl; cycloalkyl; —O-alkyl; -Oalkenyl; —O-alkynyl; —O-aryl; —CN; —OH; oxo; halo; —C(═O)OH; —C(═O)halo; —OC(═O)halo; —CF₃; N₃; NO₂; —NH₂; —NH(alkyl); —N(alkyl)₂; NH(aryl); —N(aryl)₂; —C(═O)NH₂; —C(═O)NH(alkyl); —C(═O)N(alkyl)₂; —C(═O)NH(aryl); —C(═O)N(aryl)₂; —OC(═O)NH₂; —C(═O)NH(heteroaryl); —C(═O)N(heteroaryl)₂; —NHOH; —NOH(alkyl); —NOH(aryl); —OC(═O)NH(alkyl); —OC(═O)N(alkyl)₂; —OC(═O)NH(aryl); —OC(═O)N(aryl)₂; —CHO; —C(═O)(alkyl); —C(═O)(aryl); —C(═O)O(alkyl); —C(═O)O(aryl); —OC(═O)(alkyl); —OC(═O)(aryl); —OC(═O)O(alkyl); —OC(═O)O(aryl); —S-alkyl; —S-alkenyl; —S-alkynyl; —SC(═O)₂-aryl, —SC(═O)₂-alkyl; —SC(═O)₂-alkenyl; —SC(═O)₂-alkynyl; and —SC(═O)₂-aryl; —O—S(═O)₂-alkyl, —O—S(═O)₂-alkenyl, —O—S(═O)₂-alkynyl, —O—S(═O)₂-aryl, —(CH₂)ₙ—NH₂, —(CH₂)ₙ—NH(alkyl), —(CH₂)ₙ—N(alkyl)₂, —(CH₂)ₙ—NH(aryl), or —(CH₂)ₙN(aryl)₂, wherein n is 1 to 8.

Examples of suitable substituents may include, but are not limited to: —(C₁-C₈)alkyl; —(C₁-C₈)alkenyl; —(C₁-C₈)alkynyl; phenyl; —(C₂-C₅) heteroaryl; —(C₁-C₆)heterocycloalkyl; —(C₃-C₇)cycloalkyl; —O—(C₁-C₈)alkyl; —O—(C₁-C₈)alkenyl; —O—(C₁-C₈)alkynyl; —O-phenyl; —CN; —OH; oxo; halo; —C(═O)OH; —COhalo; —OC(═O)halo; —CF₃; N₃; NO₂; —NH₂; —NH((C₁-C₈)alkyl); —N((C₁-C₈)alkyl)₂; —NH(phenyl); —N(phenyl)₂; —C(═O)NH₂; —C(═O)NH((C₁-C₈)alkyl); —C(═O)N((C₁-C₈)alkyl)₂; —C(═O)NH(phenyl); —C(═O)N(phenyl)₂; —OC(═O)NH₂; —NHOH; —NOH((C₁-C₈)alkyl); —NOH(phenyl); —OC(═O)NH((C₁-C₈)alkyl); —OC(═O)N((C₁-C₈)alkyl)₂; —OC(═O)NH(phenyl); —OC(═O)N(phenyl)₂; —CHO; —CO((C₁-C₈)alkyl); —CO(phenyl); —C(═O)O((C₁-C₈)alkyl); —C(═O)O(phenyl); —OC(═O)((C₁-C₈)alkyl); —OC(═O)(phenyl); —OC(═O)O((C₁-C₈)alkyl); —OC(═O)O(phenyl); —S—(C₁-C₈)alkyl; —S—(C₁-C₈)alkenyl; —S—(C₁-C₈)alkynyl; and —S-phenyl, —SC(O)-phenyl, —SC(O)—(C₁-C₈)alkyl, —SC(O)—(C₁-C₈)alkenyl, —SC(O)—(C₁-C₈ alkynyl), —O—S(═O)₂—(C₁-C₈)alkyl, —O—S(═O)₂—(C₁-C₈)alkenyl, —O—S(═O)₂—(C₁-C₈)alkynyl, —O—S(═O)₂-phenyl, —(CH₂)ₙNH₂, —(CH₂)ₙNH((C₁-C₈)alkyl), —(CH₂)ₙN((C₁-C₈)alkyl)₂, —(CH₂)ₙNH(aryl), or —(CH₂)ₙN(phenyl)₂, wherein n is 1 to 8. One of ordinary skill in the art may readily choose a suitable substituent based on the stability and pharmacological and synthetic activity of compounds of the present invention. A designation of (Cₓ-C_{y}) indicates the number of carbon atoms in the group, for example, (C₁-C₈) means that the group contains 1 to 8 carbon atoms.

“Synthon”: As used herein, the term “synthon” refers to a structural unit within a small molecule that can be formed and/or assembled by synthetic procedures known to one of ordinary skill in the art.

“Therapeutically effective amount”: As used herein, the term “therapeutically effective amount” means the amount of a compound that may elicit a biological or medical response in the mammal that is being that is being treated by a medical doctor or other clinician.

“Treat” “treating” and “treatment”: The terms “treat” “treating” and “treatment,” as used herein, contemplate an action that occurs while a patient is suffering from the specified disease or disorder, which reduces the severity of the disease or disorder. As used in the chemical structure drawings, the following “wavy line” indicates a bond at the point that a chemical group is attached to another chemical group:

[symbol]

Unit dosage form: The expression “unit dosage form” as used herein refers to a physically discrete unit of a provided formulation appropriate for the subject to be treated. It will be understood, however, that the total daily usage of provided formulation will be decided by the attending physician within the scope of sound medical judgment. The specific effective dose level for any particular subject or organism will depend upon a variety of factors including the disorder being treated and the severity of the disorder; activity of specific active agent employed; specific formulation employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

### DETAILED DESCRIPTION OF CERTAIN EMBODIMENTS

### 1. Description of Exemplary Compounds

Compounds provided by the present invention include those described generally above, and are further illustrated by all classes, subclasses and species of each of these compounds disclosed herein.

According to one aspect, the present invention provides compounds of the formula I:

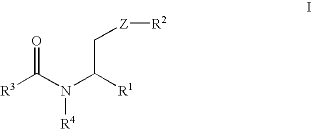

or a pharmaceutically acceptable salt, enantiomer, diastereomer, or double bond isomer thereof, wherein:

Z is —S—, —O—, —Se—, —S(O)—, —SO₂—, or —NH—;

R¹ is a heteroaryl group, or a moiety selected from

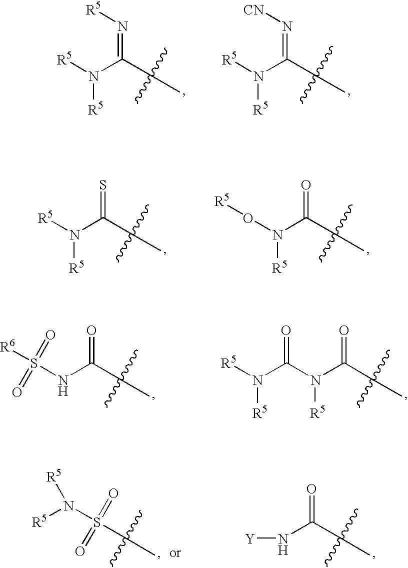

wherein at least one R⁵ group is H;

R⁵ is independently selected from H, alkyl, aryl, alkenyl, or alkynyl, wherein R⁵ is optionally substituted with one or two R⁷ groups;

R⁶ is H, alkyl, aryl, alkenyl, alkynyl, or a cyclic radical, where R⁶ is optionally substituted with one or two R⁷ groups;

Y is selected from H, —NH₂, —OH, —NH-phenyl, —NHC(O)CH₃, —NHCH₃, or —(C₁-C₈)alkyl;

R² is an aliphatic group substituted with one or more R⁷ groups;

R³ is alkoxy, aminoalkyl, alkyl, aryl, alkenyl, alkynyl, or a cyclic radical, where R³ is optionally substituted with one or two R⁷ groups;

R⁴ is H, alkyl, aryl, alkenyl, alkynyl, or a cyclic radical, where R⁴ is optionally substituted with one or two R⁷ groups; and

R⁷ is —NHC(═O)(C₁-C₈)alkyl, —(C₁-C₈)alkyl, —(C₁-C₈)alkenyl, —(C₁-C₈)alkynyl, phenyl, —(C₂-C₅)heteroaryl, —(C₁-C₆)heterocycloalkyl, —(C₃-C₇)cycloalkyl, —O—(C₁-C₈)alkyl, —O—(C₁-C₈)alkenyl, —O—(C₁-C₈)alkynyl, —O-phenyl, —CN, —OH, oxo, halo, —C(═O)OH, —COhalo, —OC(═O)halo, —CF₃, N₃, NO₂, —NH₂, —NH((C₁-C₈)alkyl), —N((C₁-C₈)alkyl)₂, —NH(phenyl), —N(phenyl)₂, —C(═O)NH₂, —C(═O)NH((C₁-C₈)alkyl), —C(═O)N((C₁-C₈)alkyl)₂, —C(═O)NH(phenyl), —C(═O)N(phenyl)₂, —OC(═O)NH₂, —NHOH, —NOH((C₁-C₈)alkyl), —NOH(phenyl), —OC(═O)NH((C₁-C₈)alkyl), —OC(═O)N((C₁-C₈)alkyl)₂, —OC(═O)NH(phenyl), ═OC(═O)N(phenyl)₂, —CHO, —CO((C₁-C₈)alkyl), —CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈) alkyl), —OC(═O)O(phenyl), —S—(C₁-C₈)alkyl, —S—(C₁-C₈)alkenyl, —S—(C₁-C₈)alkynyl, and —S-phenyl, SC(O)-phenyl, —SC(O)—(C₁-C₈)alkyl, —SC(O)—(C₁-C₈)alkenyl, —SC(O)—(C₁-C₈ alkynyl), —O—S(═O)₂—(C₁-C₈)alkyl, —O—S(═O)₂—(C₁-C₈)alkenyl, —O—S(═O)₂—(C₁-C₈)alkynyl, —O—S(═O)₂-phenyl, —(CH₂)ₙNH₂, —(CH₂)ₙ—NH((C₁-C₈)alkyl), —(CH₂)ₙN((C₁-C₈) alkyl)₂, —(CH₂)ₙNH(phenyl), or —(CH₂)ₙN(phenyl)₂, wherein n is 1 to 8.

In certain embodiments, the present invention provides a compound of formula Ia,

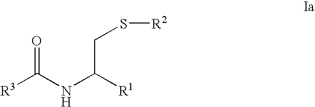

or a pharmaceutically acceptable salt, enantiomer, diastereomer, or double bond isomer thereof, wherein:

R¹ is a heteroaryl group, or a moiety selected from

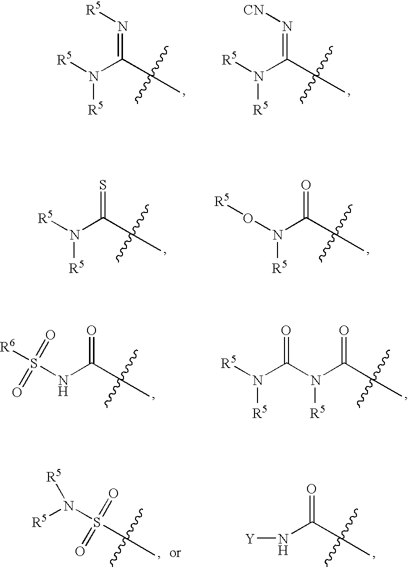

wherein at least one R⁵ group is H;

R⁵ is independently selected from H, alkyl, aryl, alkenyl, or alkynyl, wherein R⁵ is optionally substituted with one or two R⁷ groups;

R⁶ is H, alkyl, aryl, alkenyl, alkynyl, or a cyclic radical, where R⁶ is optionally substituted with one or two R⁷ groups;

Y is selected from H, —NH₂, —OH, —NH-phenyl, —NHC(O)CH₃, —NHCH₃, or -(C₁-C₈)alkyl;

R² is an aliphatic group substituted with one or more R⁷ groups;

R³ is alkoxy, aminoalkyl, alkyl, aryl, alkenyl, alkynyl, or a cyclic radical, where R³ is optionally substituted with one or two R⁷ groups; and

R⁷ is —NHC(═O)(C₁-C₈)alkyl, —(C₁-C₈)alkyl, —(C₁-C₈)alkenyl, —(C₁-C₈)alkynyl, phenyl, —(C₂-C₅)heteroaryl, —(C₁-C₆)heterocycloalkyl, —(C₃-C₇)cycloalkyl, —O—(C₁-C₈)alkyl, —O—(C₁-C₈)alkenyl, —O—(C₁-C₈)alkynyl, —O-phenyl, —CN, —OH, oxo, halo, —C(═O)OH, —COhalo, —OC(═O)halo, —CF₃, N₃, NO₂, —NH₂, —NH((C₁-C₈)alkyl), —N((C₁-C₈)alkyl)₂, —NH(phenyl), —N(phenyl)₂, —C(═O)NH₂, —C(═O)NH((C₁-C₈)alkyl), —C(═O)N((C₁-C₈)alkyl)₂, —C(—O)NH(phenyl), —C(═O)N(phenyl)₂, —OC(═O)NH₂, —NHOH, —NOH((C₁-C₈)alkyl), —NOH(phenyl), —OC(═O)NH((C₁-C₈)alkyl), —OC(═O)N((C₁-C₈)alkyl)₂, —OC(═O)NH(phenyl), ═OC(═O)N(phenyl)₂, —CHO, —CO((C₁-C₈)alkyl), —CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈) alkyl), —OC(═O)O(phenyl), —S—(C₁-C₈)alkyl, —S—(C₁-C₈)alkenyl, —S—(C₁-C₈)alkynyl, and —S-phenyl, —SC(O)-phenyl, —SC(O)—(C₁-C₈)alkyl, —SC(O)—(C₁-C₈)alkenyl, —SC(O)—(C₁-C₈ alkynyl), —O—S(═O)₂—(C₁-C₈)alkyl, —O—S(═O)₂—(C₁-C₈)alkenyl, —O—S(═O)₂—(C₁-C₈)alkynyl, —O—S(═O)₂-phenyl, —(CH₂)ₙNH₂, —(CH₂)ₙ—NH((C₁-C₈)alkyl), —(CH₂)ₙN((C₁-C₈) alkyl)₂, —(CH₂)ₙNH(phenyl), or —(CH₂)ₙN(phenyl)₂, wherein n is 1 to 8.

As defined generally above, Z is —S—, —O—, —Se—, —S(O)—, —SO₂—, or —NH—. In certain embodiments, Z is —S—. In certain embodiments, Z is —O—. In certain embodiments, Z is —Se—. In certain embodiments, Z is —S(O). In certain embodiments, Z is —SO₂—. In certain embodiments, Z is —NH—.

As defined generally above, the R¹ group of Formula I is a heteroaryl group, or a moiety selected from

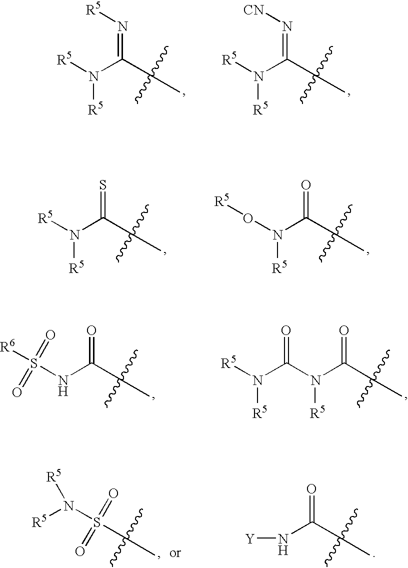

In certain embodiments, the R¹ group of Formulae I and/or Ia is a heteroaryl moiety. In certain embodiments, the heteroaryl moiety is selected from,

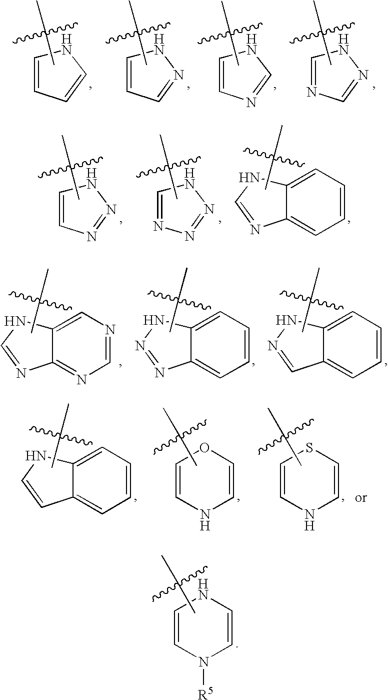

In certain embodiments, R¹ is

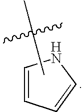

In certain embodiments, R¹ is

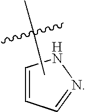

In certain embodiments, R¹ is

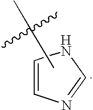

In certain embodiments, R¹ is

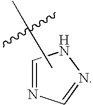

In certain embodiment, R¹ is

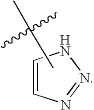

In certain embodiments, R¹ is

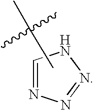

In certain embodiments, R¹ is

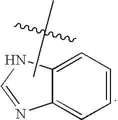

In certain embodiments, R¹ is

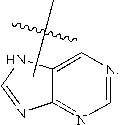

In certain embodiments, R¹

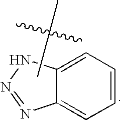

In certain embodiments, R¹ is

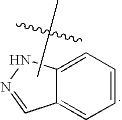

In certain embodiments, R¹ is

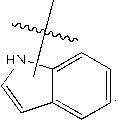

In certain embodiments, R¹ is

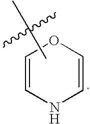

In certain embodiments, R¹ is

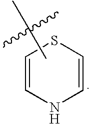

In certain embodiments, R¹ is

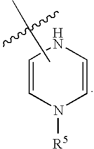

In certain embodiments, the R¹ group of Formula I is a moiety selected from

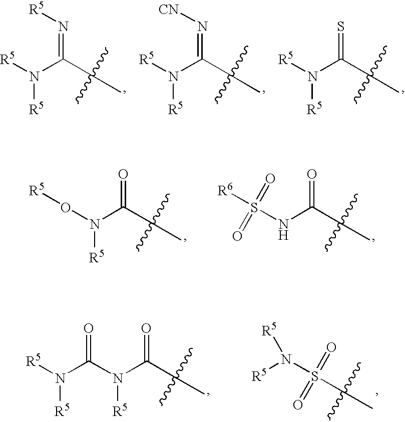

or

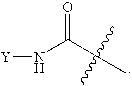

In certain embodiments, R¹ is

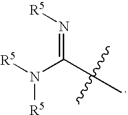

In certain embodiments, R¹ is

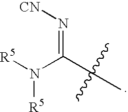

In certain embodiments, R¹ is

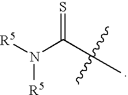

In certain embodiments, R¹ is

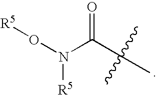

In certain embodiments, R¹ is

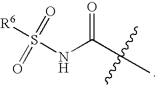

In certain embodiments, R¹ is

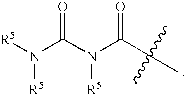

In certain embodiments, R¹ is

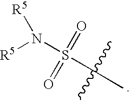

In certain embodiments, R¹ is

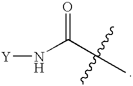

In certain embodiments R¹ is —C(O)NH—NH₂ when Y is —NH₂. In certain embodiments, R¹ is —C(O)NH—OH when Y is —OH. In certain embodiments, R¹ is —C(O)NH—NH-phenyl, when Y is —NH-Phenyl. In certain embodiments, R¹ is —C(O)NH—NHC(O)CH₃ when Y is —NHC(O)CH₃. In certain embodiments, R¹ is —C(O)NH—NHCH₃ when Y is —NHCH₃.

As defined generally above, the Y group is selected from H, —NH₂, —OH, —NH-phenyl, —NHC(O)CH₃, —NHCH₃, or —(C₁-C₈)alkyl. In certain embodiments, Y is —H. In certain embodiments, Y is —NH₂. In certain embodiments, Y is —OH. In certain embodiments, Y is —NH-phenyl. In certain embodiments, Y is —NHC(O)CH₃. In certain embodiments, Y is —NHCH₃. In certain embodiments, Y is —(C₁-C₈)alkyl. In certain embodiments, Y is —CH₃. In certain embodiments, Y is —CH₂CH₃. In certain embodiments, Y is —(CH₂)₂CH₃. In certain embodiments, Y is —(CH₂)₃CH₃. In certain embodiments, Y is —(CH₂)₄CH₃. In certain embodiments, Y is —(CH₂)₅CH₃. In certain embodiments, Y is —(CH₂)₆CH₃. In certain embodiments, Y is —(CH₂)₇CH₃.

As defined generally above, the R⁵ group is independently selected from H, alkyl, aryl, alkenyl, or alkynyl, wherein R⁵ is optionally substituted with one or two R⁷ groups. In certain embodiments, R⁵ is H. In certain embodiments R⁵ is alkyl. In certain embodiments, R⁵ is —CH₃. In certain embodiments, R⁵ is CH₂CH₃. In certain embodiments, R⁵ is —(CH₂)₂CH₃. In certain embodiments R⁵ is aryl. In certain embodiments R⁵ is alkenyl. In certain embodiments R⁵ is alkynl. In certain embodiments R⁵ is substituted with one R⁷ group. In certain embodiments R⁵ is substituted with two R⁷ groups.

As defined generally above, the R⁶ group is selected from H, alkyl, aryl, alkenyl, or alkynyl, wherein R⁶ is optionally substituted with one or two R⁷ groups. In certain embodiments, R⁶ is H. In certain embodiments R⁶ is alkyl. In certain embodiments R⁶ is alkyl. In certain embodiments, R⁶ is —CH₃. In certain embodiments, R⁶ is —CH₂CH₃. In certain embodiments, R⁶ is —(CH₂)₂CH₃. In certain embodiments R⁶ is aryl. In certain embodiments R⁶ is alkenyl. In certain embodiments R⁶ is alkynl. In certain embodiments R⁶ is a cyclic radical. In certain embodiments R⁶ is substituted with one R⁷ group. In certain embodiments R⁶ is substituted with two R⁷ groups.

As defined generally above, the R² group is an aliphatic group substituted with one or more R⁷ groups. In certain embodiments, R² is an aliphatic group substituted with one R⁷ group. In certain embodiments, R² is an aliphatic group substituted with two R⁷ groups. In certain embodiments, R² is a straight or branched chain alkyl group, with 10 to 25 carbon atoms. In certain embodiments, R² is a straight or branched chain alkenyl group, with 10 to 25 carbon atoms and one or more double bonds. In certain embodiments, R₂

is

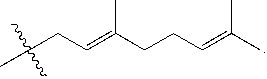

In certain embodiments, R₂ is

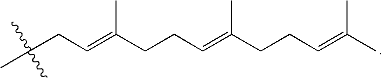

In certain embodiments, R₂ is

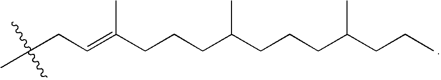

In certain embodiments, R₂ is

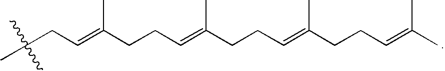

In certain embodiments, R₂
is

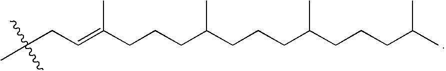

In certain embodiments, R₂ is

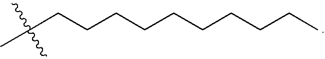

In certain embodiments, R₂ is

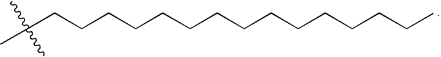

In certain embodiments, R₂ is

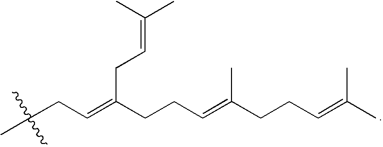

In certain embodiments, R₂ is

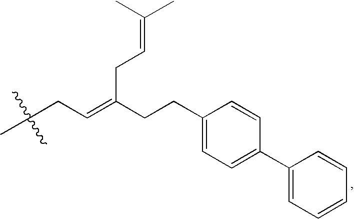

in any isomeric form, optionally substituted with one or more R⁷ groups.

As defined generally above, the R³ group of Formula I is alkoxy, aminoalkyl, alkyl, aryl, alkenyl, alkynyl, or a cyclic radical, wherein R³ is optionally substituted with one or two R⁷ groups.

In certain embodiments, R³ is alkoxy. In certain embodiments, R³ is aminoalkyl. In certain embodiments, R³ is an alkyl group optionally substituted with one or two R⁷ groups. In certain embodiments, R³ is —CH₃. In certain embodiments, R³ is —CH₂CH₃. In certain embodiments, R³ is —(CH₂)₂CH₃. In certain embodiments, R³ is aryl. In certain embodiments, R³ is alkenyl. In certain embodiments, R³ is alkynyl. In certain embodiments, R³ is a cyclic radical. In certain embodiments R³ is substituted with one R⁷ group. In certain embodiments R³ is substituted with two R⁷ groups. In certain embodiments R³ is —OC(CH₃)₃. In certain embodiments, R³ is phenyl. In certain embodiments, R³ is —(CH₂)₂C(O)OH. In certain embodiments, R³ is —CH(CH₃)NHC(O)CH₃.

As defined generally above, the R⁴ group of Formula I is H, alkyl, aryl, alkenyl, alkynyl, or a cyclic radical, where R⁴ is optionally substituted with one or two R⁷ groups.

In certain embodiments, R⁴ is H. In certain embodiments, R⁴ is an alkyl group optionally substituted with one or two R⁷ groups. In certain embodiments, R⁴ is —CH₃. In certain embodiments, R⁴ is —CH₂CH₃. In certain embodiments, R⁴ is —CH₂)₂CH₃. In certain embodiments, R⁴ is aryl. In certain embodiments, R⁴ is alkenyl. In certain embodiments, R⁴ is alkynyl. In certain embodiments, R⁴ is a cyclic radical. In certain embodiments R⁴ is substituted with one R⁷ group. In certain embodiments R⁴ is substituted with two R⁷ groups.

As defined generally above, the R⁷ group of Formula I is —NHC(═O)—(C₁-C₈)alkyl, —(C₁-C₈)alkyl, —(C₁-C₈)alkenyl, —(C₁-C₈)alkynyl, phenyl, —(C₂-C₅)heteroaryl, —(C₁-C₆)heterocycloalkyl, —(C₃-C₇)cycloalkyl, —O—(C₁-C₈)alkyl, —O—(C₁-C₈)alkenyl, —O—(C₁-C₈)alkynyl, —O-phenyl, —CN, —OH, oxo, halo, —C(═O)OH, —C(═O)halo, —OC(═O)halo, —CF₃, N₃, NO₂, —NH₂, —NH((C₁-C₈)alkyl), —N((C₁-C₈)alkyl)₂, —NH(phenyl), —N(phenyl)₂, —C(═O)NH₂, —C(═O)NH((C₁-C₈)alkyl), —C(═O)N((C₁-C₈)alkyl)₂, —C(═O)NH(phenyl), —C(═O)N(phenyl)₂, —OC(═O)NH₂, —NHOH, —NOH((C₁-C₈)alkyl), —NOH(phenyl), —OC(═O)NH((C₁-C₈)alkyl), —OC(═O)N((C₁-C₈)alkyl)₂, —OC(═O)NH(phenyl), —OC(═O)N(phenyl)₂, —CHO, —CO((C₁-C₈)alkyl), —CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈) alkyl), —OC(═O)O(phenyl), —S—(C₁-C₈)alkyl, —S—(C₁-C₈)alkenyl, —S—(C₁-C₈)alkynyl, and —S-phenyl, SC(O)-phenyl, —SC(O)—(C₁-C₈)alkyl, —SC(O)—(C₁-C₈)alkenyl, —SC(O)—(C₁-C₈ alkynyl), —O—S(═O)₂—(C₁-C₈)alkyl, —O—S(═O)₂—(C₁-C₈)alkenyl, —O—S(═O)₂—(C₁-C₈)alkynyl, —O—S(═O)₂-phenyl, —(CH₂)ₙNH₂, —(CH₂)ₙ—NH((C₁-C₈)alkyl), —(CH₂)ₙN((C₁-C₈)alkyl)₂, —(CH₂)ₙNH(phenyl), or —(CH₂)ₙN(phenyl)₂.

In certain embodiments R⁷ is —NHC(═O)—(C₁-C₈)alkyl. In certain embodiments R⁷ is —(C₁-C₈)alkyl. In certain embodiments R⁷ is —(C₁-C₈)alkenyl. In certain embodiments R⁷ is —(C₁-C₈)alkynyl. In certain embodiments R⁷ is phenyl. In certain embodiments R⁷ is —(C₂-C₅)heteroaryl. In certain embodiments R⁷ is —(C₁-C₆)heterocycloalkyl. In certain embodiments R⁷ is —(C₃-C₇)cycloalkyl. In certain embodiments R⁷ is —O—(C₁-C₈)alkyl. In certain embodiments R⁷ is —O—(C₁-C₈)alkenyl. In certain embodiments R⁷ is —O—(C₁-C₈)alkynyl. In certain embodiments R⁷ is —O-phenyl. In certain embodiments R⁷ is —CN. In certain embodiments R⁷ is —OH. In certain embodiments R⁷ is oxo. In certain embodiments R⁷ is halo. In certain embodiments R⁷ is —C(═O)OH. In certain embodiments R⁷ is —C(═O)halo. In certain embodiments R⁷ is —OC(═O)halo. In certain embodiments R⁷ is —CF₃. In certain embodiments R⁷ is N₃. In certain embodiments R⁷ is NO₂. In certain embodiments R⁷ is —NH₂. In certain embodiments R⁷ is —NH((C₁-C₈)alkyl). In certain embodiments R⁷ is —N((C₁-C₈)alkyl)₂. In certain embodiments R⁷ is —NH(phenyl). In certain embodiments R⁷ is —N(phenyl)₂. In certain embodiments R⁷ is —C(═O)NH₂. In certain embodiments R⁷ is

—C(═O)NH((C₁-C₈)alkyl). In certain embodiments R⁷ is —C(═O)N((C₁-C₈)alkyl)₂. In certain embodiments R⁷ is —C(═O)NH(phenyl). In certain embodiments R⁷ is —C(═O)N(phenyl)₂. In certain embodiments R⁷ is —OC(═O)NH₂. In certain embodiments R⁷ is —NHOH. In certain embodiments R⁷ is —NOH((C₁-C₈)alkyl). In certain embodiments R⁷ is —NOH(phenyl). In certain embodiments R⁷ is —OC(═O)NH((C₁-C₈)alkyl). In certain embodiments R⁷ is —OC(═O)N((C₁-C₈)alkyl)₂. In certain embodiments R⁷ is —OC(═O)NH(phenyl). In certain embodiments R⁷ is —OC(═O)N(phenyl)₂. In certain embodiments R⁷ is —CHO. In certain embodiments R⁷ is —CO((C₁-C₈)alkyl). In certain embodiments R⁷ is —CO(phenyl). In certain embodiments R⁷ is —C(═O)O((C₁-C₈)alkyl). In certain embodiments R⁷ is —C(═O)O(phenyl). In certain embodiments R⁷ is —OC(═O)((C₁-C₈)alkyl). In certain embodiments R⁷ is —OC(═O)(phenyl). In certain embodiments R⁷ is —OC(═O)O((C₁-C₈) alkyl). In certain embodiments R⁷ is —OC(═O)O(phenyl). In certain embodiments R⁷ is —S—(C₁-C₈)alkyl. In certain embodiments R⁷ is —S—(C₁-C₈)alkenyl. In certain embodiments R⁷ is
—S—(C₁-C₈)alkynyl. In certain embodiments R⁷ is —S-phenyl. In certain embodiments R⁷ is —SC(O)-phenyl. In certain embodiments R⁷ is —SC(O)—(C₁-C₈)alkyl. In certain embodiments R⁷ is —SC(O)—(C₁-C₈)alkenyl. In certain embodiments R⁷ is —SC(O)—(C₁-C₈ alkynyl). In certain embodiments R⁷ is —O—S(═O)₂—(C₁-C₈)alkyl. In certain embodiments R⁷ is —O—S(═O)₂—(C₁-C₈)alkenyl. In certain embodiments R⁷ is —O—S ═O)₂—(C₁-C₈)alkynyl. In certain embodiments R⁷ is
—O—S(═O)₂-phenyl. In certain embodiments R⁷ is —(CH₂)ₙNH₂. In certain embodiments R⁷ is —(CH₂)ₙ—NH((C₁-C₈)alkyl). In certain embodiments R⁷ is —(CH₂)ₙN((C₁-C₈)alkyl)₂. In certain embodiments R⁷ is —(CH₂)ₙNH(phenyl). In certain embodiments R⁷ is —(CH₂)ₙN(phenyl)₂.

As defined generally above, n is 1 to 8. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8.

Exemplary compounds of the present invention are set forth in Table 1 below.

TABLE 1
Exemplary Compounds

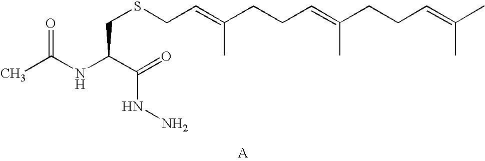

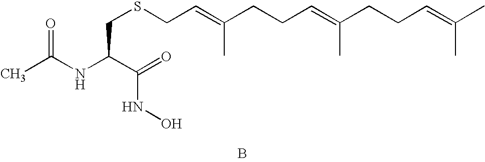

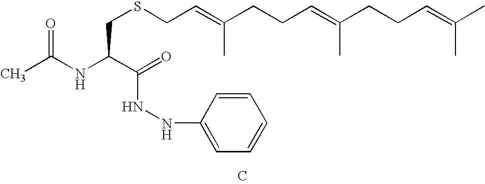

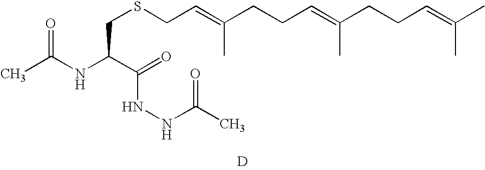

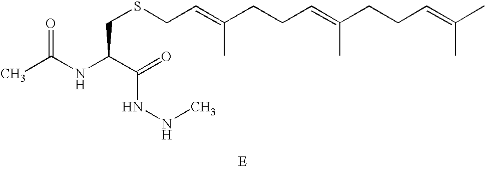

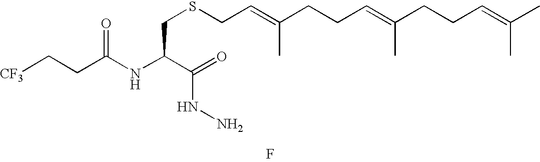

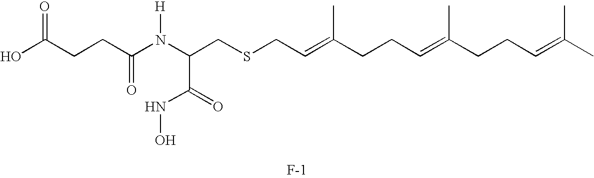

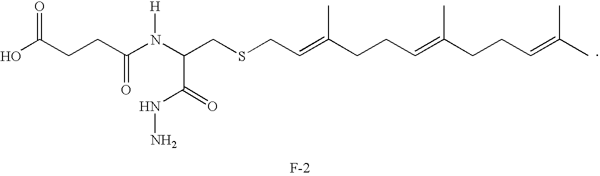

In certain embodiments, the present invention provides any compound depicted in Table 1, above, or a pharmaceutically acceptable salt thereof.

Additional exemplary compounds of the present invention are set forth in Table 2 below.

TABLE 2
Exemplary Compounds

| | | | Exact Mass |
|---|---|---|---|
| Compound | Structure | IUPAC Name | (Da) |
| G-1 | 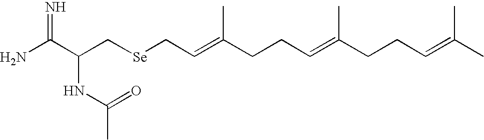 | -N-(1-carbamimidoyl- 2-{[3,7,11- trimethyldodeca- 2,6,10-trien-1- yl]selanyl}ethyl) acetamide | 413.19 |
| G-2 | 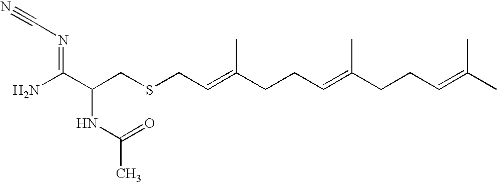 | -N-[1-(N′- cyanocarbamimidoyl)- 2-{[3,7,11- trimethyldodeca-2,6, 10-trien-1 yl]sulfanyl}ethyl]acetamide | 390.25 |
| G-3 | 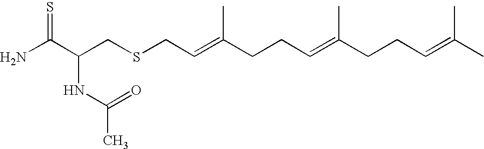 | -N-(1-carbamothioyl-2- {[3,7,11- trimethyldodeca- 2,6,10-trien-1- yl]sulfanyl}ethyl) acetamide | 382.21 |
| G-4 | 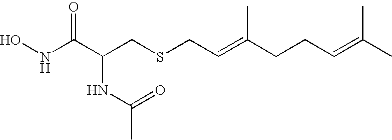 | -3-{[3,7-dimethylocta- 2,6-dien-1- yl]sulfanyl}-2- acetamido-N- hydroxypropanamide | 314.17 |
| G-5 | 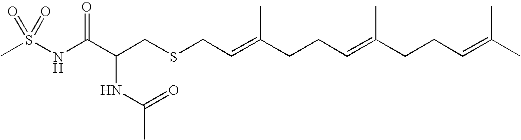 | -2-acetamido-N- methanesulfonyl-3-{[3, 7,11-trimethyldodeca- 2,6,10-trien-1- yl]sulfanyl}propanamide | 444.21 |
| G-6 | 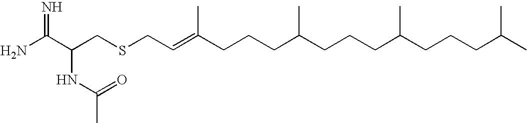 | N-(1-carbamimidoyl-2- {[3,7,11,15- tetramethylhexadec-2- en-1-yl]sulfanyl}ethyl)acetamide | 439.36 |
| G-7 | 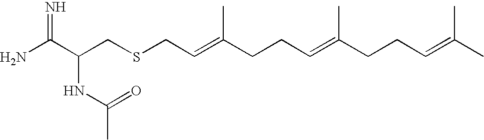 | N-(1-carbamimidoyl-2- {[3,7,11- trimethyldodeca- 2,6,10-trien-1- yl]sulfanyl}ethyl)acetamide | 365.25 |
| G-8 | 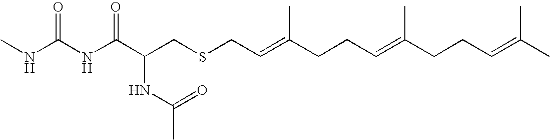 | N-{1- [(methylcarbamoyl)amino]- 1-oxo-3-{[3,7,11- trimethyldodeca- 2,6,10-trien-1- yl]sulfanyl}propan-2- yl}acetamide | 423.26 |
| G-9 | 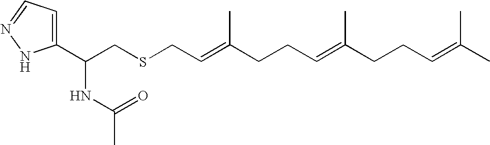 | N-[1-(1H-pyrazol-5- yl)-2-{[3,7,11- trimethyldodeca- 2,6,10-trien-1- yl]sulfanyl}ethyl]acetamide | 389.25 |
| G-10 | 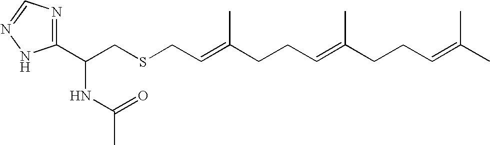 | N-[1-(1H-1,2,4-triazol- 5-yl)-2-{[3,7,11- trimethyldodeca- 2,6,10-trien-1- yl]sulfanyl}ethyl]acetamide | 390.25 |
| G-11 | 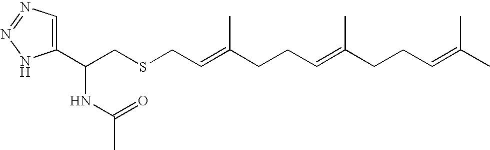 | -N-[1-(1H-1,2,3,- triazol-5-yl)-2-{(3,7,11- trimethyldodeca- 2,6,10-trien-1- yl)sulfanyl}ethyl]acetamide | 390.25 |
| G-12 | 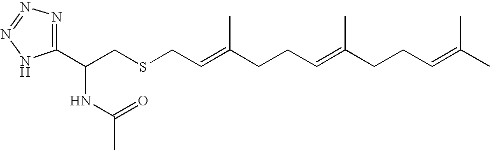 | N-[1-(1H-1,2,3,4- tetrazol-5-yl)-2- [(3,7,11- trimethyldodeca- 2,6,10-trien-1- yl)sulfanyl]ethyl]acetamide | 391.24 |
| G-13 | 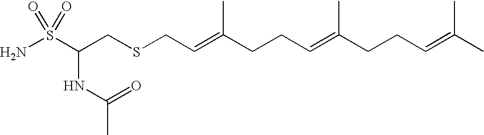 | N-{1-sulfamoyl-2- [(3,7,11- trimethyldodeca- 2,6,10-trien-1- yl)sulfanyl]ethyl}acetamide | 402.20 |
| G-14 | 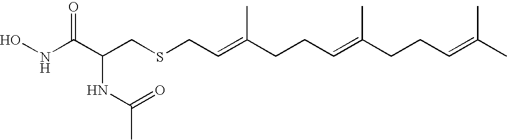 | 2-acetamido-N- hydroxy-3-[(3,7,11- trimethyldodeca- 2,6,10-trien-1- yl)sulfanyl]propanamide | 382.23 |
| G-15 | 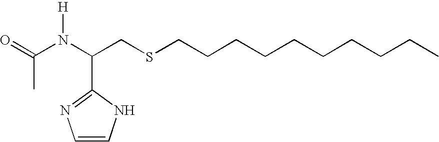 | N-[2-(decylsulfanyl)-1- (1H-imidazol-2- yl)ethyl]acetamide | 325.22 |
| G-16 | 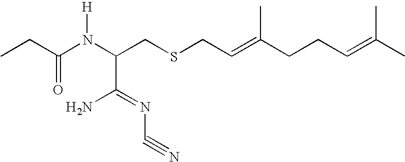 | -N-[1-(N′- cyanocarbamimidoyl)- 2-{[3,7-dimethylocta- 2,6-dien-1- yl]sulfanyl}ethyl]propanamide | 336.20 |
| G-17 | 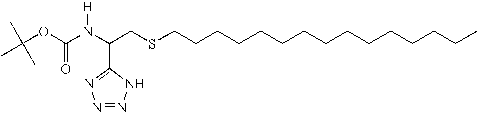 | tert-butyl-N-[2- (hexadecylsulfanyl-1- (1H-1,2,3,4-tetrazol-5- yl)ethyl]carbamate | 469.35 |
| G-18 | 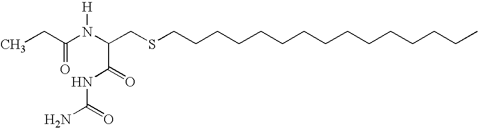 | N-[1-(carbamoylamino- 3-(hexadecylsulfanyl)- 1-oxopropan-2- yl]propanamide | 443.32 |
| G-19 | 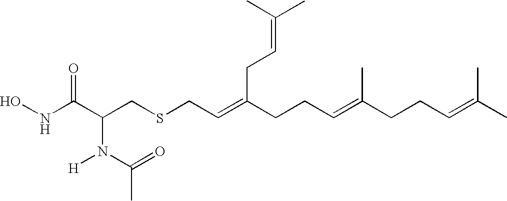 | 3-{[7,11-dimethyl-3-(3- methylbut-2-en-1- yl)dodeca-2,6,10-trien- 1-yl]sulfanyl}-2- acetamido-N- hydroxypropanamide | 436.28 |
| G-20 | 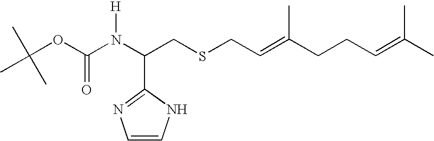 | tert-butyl N-(2-{[3,7- dimethylocta-2,6-dien-1- yl]sulfanyl}-1-(1H- imidazol-2- yl)ethyl)carbamate | 379.23 |
| G-21 | 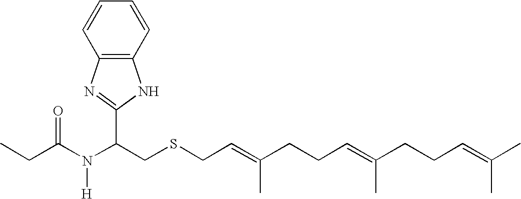 | N-1{1-H-1,3- benzodiazol-2- yl)-2{[3,7,11- trimethyldodeca-2,6, 10-trien-1- yl]sulfanyl}ethyl] propanamide | 453.28 |
| G-22 | 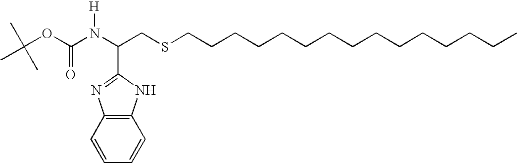 | tert-butyl N-[1-(1H-1, 3-benzodiazol-2-yl)-2- (hexadecylsulfanyl) ethyl]carbamate | 517.37 |
| G-23 | 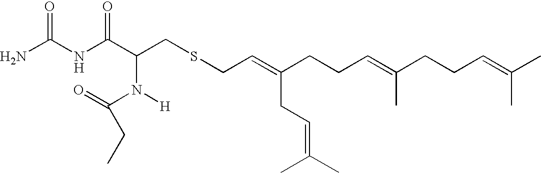 | N-[1- (carbamoylamino)-3- {[7,11-dimethyl-3- (3-methylbut-2-en-1- yl)dodeca-2,6,10- trien-1-yl]sulfanyl}-1- oxopropan-2- Y1]propanamide | 477.30 |
| G-24 | 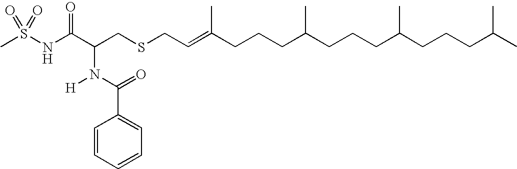 | N-methanesulfonyl-2- (phenylformanido)-3- [(3,7,11,15- tetramethylhexadec- 2-en-1-yl)sulfanyl] propanamide | 580.34 |
| G-25 | 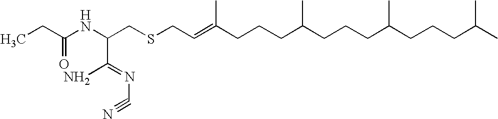 | N-[1-N′- Cyanocarbamimidoyl)- 2-[(3,7,11,15- tetramethylhexadec-2- en-1-yl)sulfanyl]ethyl]- propanamide | 478.37 |
| G-26 | 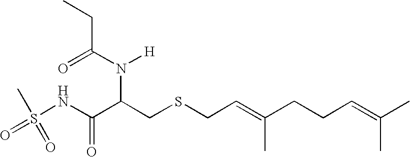 | 3-[(3,7-dimethylocta-2, 6-dien-1-yl)sulfanyl]- N-methanesulfonyl-2- propanamidopropanamide | 390.16 |
| G-27 | 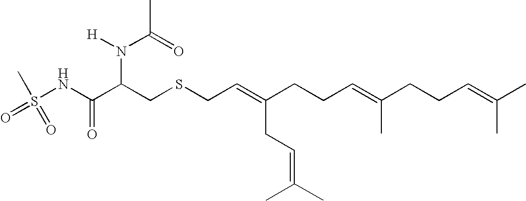 | 3-{[7,11-dimethyl-3- (3-methylbut-2-en-1- yl) dodeca- 2,6,10-trien-1- yl]sulfanyl}-2- acetamido-N- methanesulfonylpropanamide | 498.26 |
| G-28 | 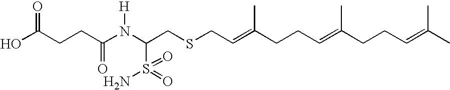 | 3-[(1-sulfamoyl-2-{[3, 7,11-trimethyldodeca- 2,6,10-trien-1- yl]sulfanyl}ethyl)carba- moyl]propanoic acid | 460.21 |
| G-29 | 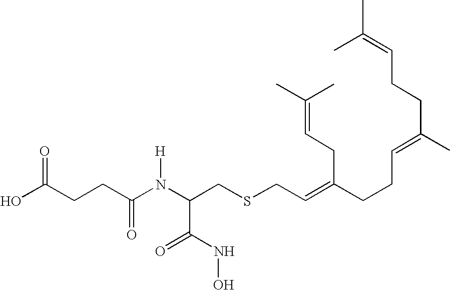 | 3-[(2-{[7,11-dimethyl- 3-(3-methylbut-2-en-1- yl) dodeca- 2,6,10-trien-1- yl]sulfanyl}-1- (hydroxycarbamoyl) ethyl) carbamoyl]propanoic acid | 494.28 |
| G-30 | 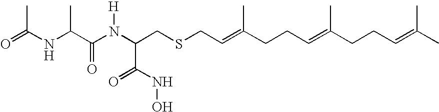 | 2-(2-acetamidopropan- amido)-N-hydroxy-3- {[3,7,11- trimethyldodeca-2,6, 10-trien-1- yl]sulfanyl}propanamide | 453.27 |
| G-31 | 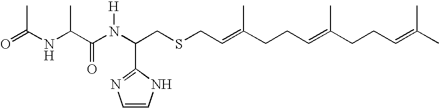 | 2-acetamido-N-[1- (1H-imidazol-2-yl)-2- {[3,7,11- trimethyldodeca-2,6, 10-trien-1- yl]sulfanyl}ethyl]propanamide | 460.29 |
| G-32 | 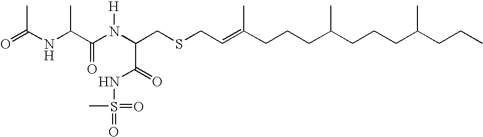 | 2-(2-acetamidopropan- amido)-N- methanesulfonyl-2- {[3,7,11,15- tetramethylhexadedc- 2-en-1-yl]sulfanyl} propanamide | 589.36 |

Compounds of the present invention include enantiomers, diastereomers, and double bond isomers of formulae I and/or Ia.

Compounds described herein (e.g., compounds of formulae I and/or Ia) may be provided according to the present invention in any of a variety of useful forms, for example as pharmaceutically acceptable salts, as particular crystal forms, etc. In some embodiments, prodrugs of such compounds are provided. Various forms of prodrugs are known in the art, for example as discussed in Bundgaard (ed.), Design of Prodrugs, Elsevier (1985); Widder et al (ed.), Methods in Enzymology, vol. 4, Academic Press (1985); Kgrogsgaard-Larsen et al. (ed.); “Design and Application of Prodrugs”, Textbook of Drug Design and Development, Chapter 5, 113-191 (1991); Bundgaard et al., Journal of Drug Delivery Reviews, 8:1-38 (1992); Bundgaard et al., J. Pharmaceutical Sciences, 77:285 et seq. (1988); and Higuchi and Stella (eds.), Prodrugs as Novel Drug Delivery Systems, American Chemical Society (1975).

Compounds of the present invention may contain one or more chiral centers and/or double bonds. Unless otherwise stated, structures depicted herein are also meant to include all isomeric (e.g., double bond isomers, enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of compounds of the present invention are within the scope of the invention, whether as distinct tautomers or a mixture of tautomers.

Unless otherwise indicated, the chemical structures depicted herein may encompass the racemic form of compounds as well as all enantiomers and stereoisomers, that is, both the stereomerically pure form (e.g., geometrically pure, enantiomerically pure, or diastereomerically pure) and enantiomeric and stereoisomeric mixtures.

A compound may be considered optically active or enantiomerically pure (i.e., substantially the R-form or substantially the S-form) with respect to a chiral center when a compound is about 90% ee (enantiomeric excess) or greater, preferably, equal to or greater than 95% ee with respect to a particular chiral center. A compound may be considered to be in enantiomerically enriched form when a compound has an enantiomeric excess of greater than about 80% ee, preferably greater than about. As used herein, a racemic mixture means about 50% of one enantiomer and about 50% of its corresponding enantiomer relative to all chiral centers in the molecule. Thus, compounds of the present invention may encompass all enantiomerically pure, enantiomerically enriched, and racemic mixtures.

Enantiomeric and stereoisomeric mixtures may be resolved into their component enantiomers or stereoisomers by well known methods, such as chiral-phase gas chromatography, chiral-phase high performance liquid chromatography, crystallizing a compound as a chiral salt complex, or crystallizing a compound in a chiral solvent or by enzymatic resolution of a compound, its precursor or its derivative. Enantiomers and stereoisomers may also be obtained from stereomerically or enantiomerically pure intermediates, reagents, and catalysts by well-known asymmetric synthetic methods.

Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C— or ¹⁴C-enriched carbon are within the scope of this invention. Such compounds are useful, for example, as analytical tools, as probes in biological assays, or as therapeutic agents in accordance with the present invention. In some embodiments, the R¹ group of formulae I and/or Ia comprises one or more deuterium atoms. Mixtures of isomeric forms may be separated and/or purified by techniques as would be known to one skilled in this art, including but not limited to column chromatography.

In certain embodiments, provided compounds modulate a G-protein signaling cascade. In certain embodiments, provided compounds inhibit inflammation. In certain embodiments, activity of provided compounds may be characterized using a variety of in vivo or in vitro assays. For example, ability of provided compounds to inhibit inflammation may be assessed, for example, using assays that assess edema, erythema, and/or inhibition of myeloperoxidase (“MPO”) as described, for example, in Example 15.

In certain embodiments, provided compounds are considered to be inhibitors of inflammation when they show a percent inhibition in an edema assay of at least about 30, 35, 40, 50, 60, 70, 80, 90 or 95%, for example when provided at a dose 0.8 mg/20 μL. In certain embodiments, provided compounds are considered to be inhibitors of inflammation when they show a percent inhibition in an edema assay of at least about 5, 10, 15, 20, 25, 30, 35, 40, 50, 60, 70, or 80%, for example when provided at a dose of 0.2 mg/20 μL.

In certain embodiments, provided compounds are considered to be inhibitors of inflammation when they show a percent inhibition in an erythema assay of at least about 25, 30, 35, 40, 50, 60, 70, 80, 90 or 95%, for example when provided at a dose of 0.8 mg/20 μL. In certain embodiments, provided compounds are considered to be inhibitors of inflammation when they show a percent inhibition in an erythema assay of at least about 5, 10, 15, 20, 25, 30, 35, 40, 50, 60, 70, 80, 90 or 95%, for example when provided at a dose of 0.2 mg/20 μL.

In certain embodiments, provided compounds are considered to be inhibitors of inflammation when they show a percent inhibition in an MPO assay of at least about 60, 70, 80, 90 or 95%, for example when provided at a dose of 0.8 mg/20 μL. In certain embodiments, provided compounds are considered to be inhibitors of inflammation when they show a percent inhibition in an MPO assay of at least about 5, 10, 15, 20, 25, 30, 35, 40, 50, 60, 70, or 80%, for example when provided at a dose of 0.2 mg/20 μL.

### 2. Methods of Syntheses

The present invention provides methods of preparing compounds provided herein. As will be appreciated by one of skill in the art, the synthetic methods described herein may be modified without departing from the scope of the present invention. For example, different starting materials and/or different reagents may be used in the inventive synthetic methods.

Exemplary methods of syntheses are illustrated in Schemes 1-5. Starting materials useful for preparing compounds and intermediates therefore, are commercially available or may be prepared from commercially available materials using known synthetic methods and reagents.

Protecting groups utilized herein typically denote groups which generally may not be found in the final therapeutic compounds but which may intentionally be introduced at some stage of the synthesis in order to protect groups which otherwise might be altered in the course of chemical manipulations. Such protecting groups may be removed or converted to the desired group at a later stage of the synthesis and compounds bearing such protecting groups thus may be of importance primarily as chemical intermediates (although some derivatives also exhibit biological activity). Accordingly, the precise structure of the protecting group is not critical. Numerous reactions for the formation and removal of such protecting groups are described in a number of standard works including, for example, “Protective Groups in Organic Chemistry”, Plenum Press, London and New York, 1973; Greene, Th. W. “Protective Groups in Organic Synthesis”, Wiley, New York, 1981; “The Peptides”, Vol. I, Schroder and Lubke, Academic Press, London and New York, 1965; “Methoden der organischen Chemie”, Houben-Weyl, 4th Edition, Vol. 15/I, Georg Thieme Verlag, Stuttgart 1974, the disclosures of which are incorporated herein by reference.

Scheme 1 below illustrates, general methodology for the general synthesis of compounds of the formulae I and/or Ia.

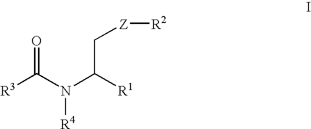

Z is —S—, A, —Se—, —S(O)—, —SO₂—, or —NH—;

R¹ is a heteroaryl group, or a moiety selected from

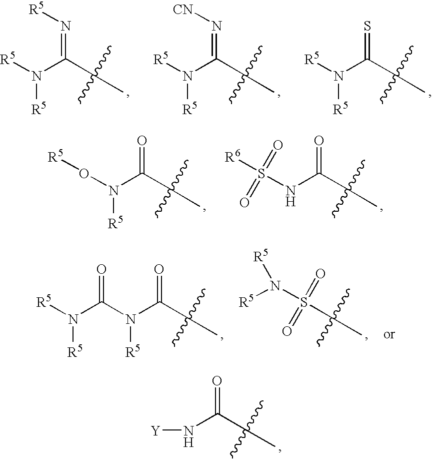

wherein at least one R⁵ group is H, and,

R⁵ is independently selected from H, alkyl, aryl, alkenyl, or alkynyl, wherein R⁵ is optionally substituted with one or two R⁷ groups;

R⁶ is H, alkyl, aryl, alkenyl, alkynyl, or a cyclic radical, where R⁶ is optionally substituted with one or two R⁷ groups;

Y is selected from H, —NH₂, —OH, —NH-phenyl, —NHC(O)CH₃, —NHCH₃, or —(C₁-C₈)alkyl;

R² is an aliphatic group substituted with one or more R⁷ groups;

R³ is alkoxy, aminoalkyl, alkyl, aryl, alkenyl, alkynyl, or a cyclic radical, where R³ is optionally substituted with one or two R⁷ groups;

R⁴ is H, alkyl, aryl, alkenyl, alkynyl, or a cyclic radical, where R⁴ is optionally substituted with one or two R⁷ groups; and

R⁷ is —NHC(═O)(C₁-C₈)alkyl, —(C₁-C₈)alkyl, —(C₁-C₈)alkenyl, —(C₁-C₈)alkynyl, phenyl,

—(C₂-C₅)heteroaryl, —(C₁-C₆)heterocycloalkyl, —(C₃-C₇)cycloalkyl, —O—(C₁-C₈)alkyl, —O—(C₁-C₈)alkenyl, —O—(C₁-C₈)alkynyl, —O-phenyl, —CN, —OH, oxo, halo, —C(═O)OH, —COhalo, —OC(═O)halo, —CF₃, N₃, NO₂, —NH₂, —NH((C₁-C₈)alkyl), —N((C₁-C₈)alkyl)₂, —NH(phenyl), —N(phenyl)₂, —C(═O)NH₂, —C(═O)NH((C₁-C₈)alkyl), —C(═O)N((C₁-C₈)alkyl)₂, —C(═O)NH(phenyl), —C(═O)N(phenyl)₂, —OC(═O)NH₂, —NHOH, —NOH((C₁-C₈)alkyl), —NOH(phenyl), —OC(═O)NH((C₁-C₈)alkyl), —OC(═O)N((C₁-C₈)alkyl)₂, —OC(═O)NH(phenyl), ═OC(═O)N(phenyl)₂, —CHO, —CO((C₁-C₈)alkyl), —CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈) alkyl), —OC(═O)O(phenyl), —S—(C₁-C₈)alkyl, —S—(C₁-C₈)alkenyl, —S—(C₁-C₈)alkynyl, and —S-phenyl, SC(O)-phenyl, —SC(O)—(C₁-C₈)alkyl, —SC(O)—(C₁-C₈)alkenyl, —SC(O)—(C₁-C₈ alkynyl), —O—S(═O)₂—(C₁-C₈)alkyl, —O—S(═O)₂—(C₁-C₈)alkenyl, —O—S(═O)₂—(C₁-C₈)alkynyl, —O—S(═O)₂-phenyl, —(CH₂)ₙNH₂, —(CH₂)ₙ—NH((C₁-C₈)alkyl), —(CH₂)ₙN((C₁-C₈) alkyl)₂, —(CH₂)ₙNH(phenyl), or —(CH₂)ₙN(phenyl)₂, wherein n is 1 to 8.

In certain embodiments, the inventive compounds are prepared as shown in Scheme 1 below.

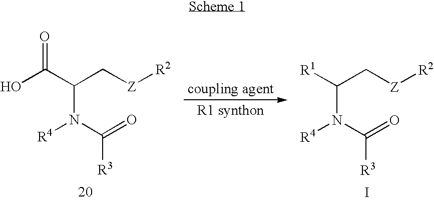

One method of synthesizing compound of formulae I and/or Ia, compound 20 may be coupled to an R¹ synthon by using a coupling agent under appropriate conditions. For example, in one embodiment compound 20 may be coupled to the R¹ synthon by adopting the methods and reagents described in M. P. Cava and M. I. Levinson, Tetrahedron, 1985, 41, 5061-5087; Rachita and Slough, Tetrahedron Letters, 1993, 34, 6821-24; Ishizuka, et al., Synthesis, 2000, 784-88; Johnson and Widlanski, Tetrahedron Letters, 42, 3677-79; Ishizuka, et al., Synthesis, 2000, 784-88; Kumar, et al., JOC, 1996, 4462-65, Koguro, et al., Synthesis, 1998, 910-914; and Racane, et al., Monatschefte fur Chemie, 2006, 137, 1571-1577, which references are hereby incorporated herein by reference.

Suitable carboxylic coupling agents may include, but are not limited to, —O-(7-azabenzotriazol-1-yl)-N,N,N′,N′-tetramethyluronium hexafluorophosphate (HATU), N,N′-dicyclohexylcarbodiimide (DCC), N-Ethyl-N′—(3-dimethylaminopropyl)carbodiimide (EDC), 1,1′-carbonyldiimidazole (CDI), (benzotriazol-1-yloxy)tripyrrolidinophosphonium hexafluorophosphate (PyBop), N,N′-diisopropylcarbodiimide (DIC)/1-hydroxybenzotriazole (HOBt), N,N,N′,N′-Tetramethyl-O-(1H-benzotriazol-1-yl)uronium hexafluorophosphate (HBTU).

The product I may be purified according to well known methods such as chromatography, and the product analyzed by low resolution mass spectrometry and NMR.

In certain embodiments, when R¹ is an aromatic heterocyclic group, compounds of formulae I and/or Ia may be prepared according to the method of Scheme 2 below.

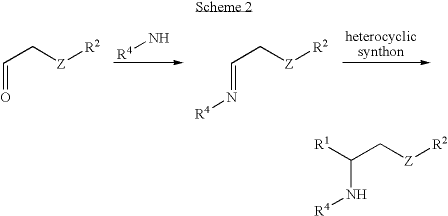

In one exemplary method, compound may be reacted with the heterocyclic synthon by adopting the synthetic methods described in Kim, et al. Bioorganic and Medicinal Chemistry Letters 14 (2004) 4651-4, or Moulin, et al. Synthesis—Stuttgart 17 (2007) 2667-73, which references are hereby incorporated herein by reference. The R3 group as added as shown in scheme 3 to yield a compound of formulae I and/or Ia.

Examples of suitable R¹ groups are shown below. These may be made with synthons such as NaN₃, TMS, etc. In certain embodiments, the R¹ group is selected from:

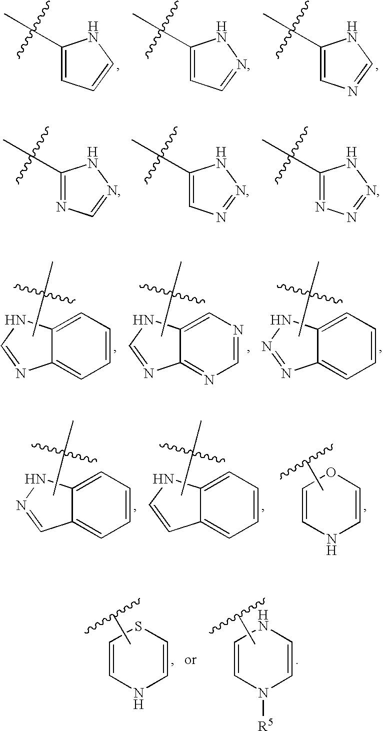

In certain embodiments, the inventive compounds are prepared as shown in Scheme 3 below.

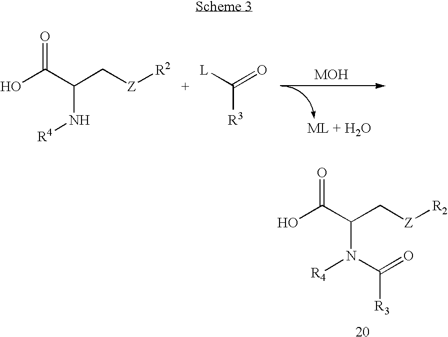

Compound 20 may be prepared according to the method set forth in Scheme 3 where M may be a metal such Na or K, and L is a leaving group such as Cl or OC(═O)R³. This reaction may be carried out, for example, in THF as solvent with K₂CO₃ as base at 50 with stirring.

In certain embodiments, the inventive compounds are prepared as shown in Scheme 4 below.

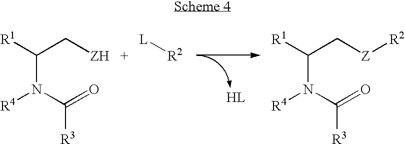

A C₁₀ to C₂₅ R group may be added according to the method of Scheme 4 where M may be a metal such Na or K, and L is a suitable leaving group such as Br or Cl.

For example, farnesol may be converted into corresponding farnesyl bromide by reaction with one half equivalent of PBr₃ in the presence of base, stirred at 0° C. for 1 h. The same method, or with substitution of PCl3, may be used to generate an activated lipid starting from an alcohol, for example, from phytol, or geranylgeraniol.

Cysteine hydrochloride, selenocysteine, and derivatives or related compounds containing a thiol or selenol may be reacted with the brominated or chlorinated lipid in ethanol as solvent with K₂CO₃ as base at RT with stirring for 3 hrs.

In certain embodiments, when R¹ is

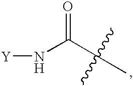

compounds of formulae I and/or Ia may be prepared according to the method of Scheme 5 below.

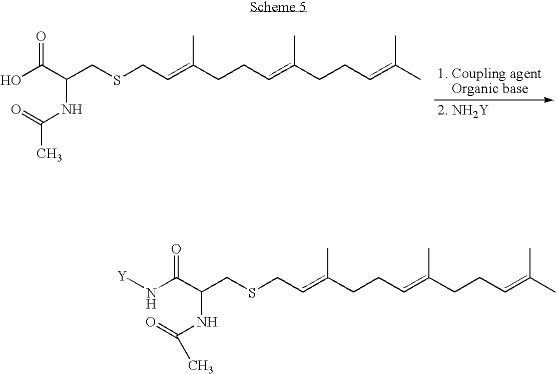

N-acetyl-5-farnesyl-L-cysteine is treated with a suitable base and suitable coupling agent, followed by the addition of a substituted hydrazine, to form a compound of formula I and/or Ia. In certain embodiments, the suitable base used is triethylamine. In certain embodiments, the suitable base used is diisopropylethylamine (DIEA). In certain embodiments, the suitable base used is 1,4-diazabicyclo[2.2.2]octane (DABCO). In certain embodiments, the suitable base used is pyridine. Exemplary carboxylic coupling agents may include, but are not limited to, 2-(1H-7-azabenzotriazol-1-yl)-1,1,3,3-tetramethyl uronium hexafluorophosphate (HATU), N,N′-dicyclohexylcarbodiimide (DCC), N-Ethyl-N′—(3-dimethylaminopropyl)carbodiimide (EDC), 1,1′-carbonyldiimidazole (CDI), (benzotriazol-1-yloxy)tripyrrolidinophosphonium hexafluorophosphate (PyBop), N,N′-diisopropylcarbodiimide (DIC)/1-hydroxybenzotriazole (HOBt), and N,N,N′,N′-Tetramethyl-O-(1H-benzotriazol-1-yl)uronium hexafluorophosphate (HBTU). In certain embodiments, the suitable carboxylic coupling agent is HATU. The reaction is typically performed in a suitable solvent to form a compound of formula I and/or Ia. In certain embodiments, the suitable solvent is a mixture of polar, aprotic solvents. In certain embodiments, whether used alone or as part of a mixture, the polar, aprotic solvents include DMF, DCM, NMP, THF, dioxane, glyme, diglyme, dichloroethane, etc.

In certain embodiments, when R¹ is

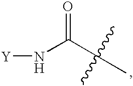

compounds of formulae I and/or Ia may be prepared according to the method of Scheme 6 below.

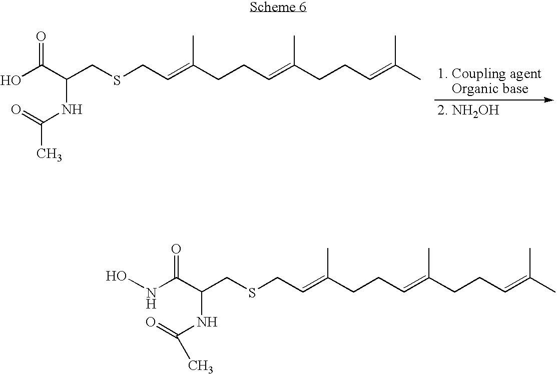

N-acetyl-5-farnesyl-L-cysteine is treated with a suitable base and a suitable coupling agent, followed by the addition of hydroxylamine, to form a compound of formula I and/or Ia. In certain embodiments, the suitable base used is triethylamine. In certain embodiments, the suitable base used is diisopropylethylamine (DIEA). In certain embodiments, the suitable base used is 1,4-diazabicyclo[2.2.2]octane (DABCO). In certain embodiments, the suitable base used is pyridine. Exemplary carboxylic coupling agents may include, but are not limited to, 2-(1H-7-azabenzotriazol-1-yl)-1,1,3,3-tetramethyl uronium hexafluorophosphate (HATU), N,N′-dicyclohexylcarbodiimide (DCC), N-Ethyl-N′—(3-dimethylaminopropyl)carbodiimide (EDC), 1,1′-carbonyldiimidazole (CDI), (benzotriazol-1-yloxy)tripyrrolidinophosphonium hexafluorophosphate (PyBop), N,N′-diisopropylcarbodiimide (DIC)/1-hydroxybenzotriazole (HOBt), and N,N,N′,N′-Tetramethyl-O-(1H-benzotriazol-1-yl)uronium hexafluorophosphate (HBTU). In certain embodiments, the suitable carboxylic coupling agent is HATU. The reaction is typically performed in a suitable solvent to form a compound of formula I and/or Ia. In certain embodiments, the suitable solvent is a mixture of polar, aprotic solvents. In certain embodiments, whether used alone or as part of a mixture, the polar, aprotic solvents include DMF, DCM, NMP, THF, dioxane, glyme, diglyme, dichloroethane, etc.

In the above-described scheme and/or steps, the R¹, R², R³, R⁴, M, L, ML, Y and Z groups of the various formulae are as described herein.

### 3. Uses

As described herein, the present invention relates to treating or lessening the severity of one or more diseases in which protein inhibitors that modulate the G-protein signaling cascade are known to play a role. Specifically, the present invention relates to a method of treating or lessening the severity of inflammatory diseases or disorders selected from inflammation (acute or chronic), inflammatory diseases or disorders (e.g., asthma, autoimmune diseases, and COPD including emphysema, chronic bronchitis and small airways disease, etc.), inflammatory responses of the immune system, skin diseases (e.g., reducing acute skin irritation for patients suffering from rosacea, atopic dermatitis, seborrheic dermatitis, psoriasis), irritable bowel syndrome (e.g., Chron's disease and ulcerative colitis, etc.), and Parkinson's disease, wherein the method comprises administering to a patient in need thereof a composition of the present invention.

In certain embodiments, provided compounds of the present invention are capable of effectively inhibiting inflammatory responses that are mediated by G-proteins or GPCRs in neutrophils, macrophages and platelets. Thus, provided compounds are inhibitors of edema, erythema and myeloperoxidase and are therefore useful for treating one or more disorders associated with inflammatory diseases or disorders as described herein. In particular, the present invention encompasses the finding that certain compounds having superior in vivo activity than other compounds in the same class. For example, compound A, compound B, compound C, compound D, compound E, and compound F show edema inhibition, erythema inhibition and MPO (myeloperoxidase) inhibition. Therefore, such compounds are administered to a subject suffering from or susceptible to one or more inflammatory diseases or disorders.

In certain embodiments, the treatment of inflammatory diseases or disorders is achieved using compounds without having the side effects of corticosteroids or NSAIDS.

In certain embodiments, such compounds are administered in vitro. In certain embodiments such compounds are administered in vivo.

Another aspect of the present invention is directed to methods of treating, preventing, or ameliorating inflammation by administering an effective amount of a provided compound.

In some embodiments, one or more inventive compounds, alone or together with one or more other pharmaceutically active agents, is used to whiten skin. In some such embodiments, the compound is applied topically.

In general, the actual quantity of provided compounds of the invention administered to a patient will vary depending on the severity and type of indication, the mode of administration, the particular compound used, the formulation used, and the response desired.

The dosage for treatment is administration, by any of the foregoing means or any other means known in the art, of an amount sufficient to bring about the desired therapeutic effect. Thus, an effective amount includes an amount of a provided compound (or mixture of provided compounds) or pharmaceutical composition of this invention that is sufficient to induce a desired effect, including specifically an anti-inflammation effect. In general, provided compounds of the present invention are highly active. For example, a provided compound can be administered at about 10 μg/kg to about 50 mg/kg body weight, depending on the specific provided compound selected, the desired therapeutic response, the route of administration, the formulation and other factors known to those of skill in the art.

### 4. Compositions and Formulations

Compounds of the present invention may be formulated into pharmaceutical compositions with at least one compound described herein together with one or more pharmaceutically acceptable carriers, including but not limited to excipients, such as diluents, carriers and the like, and additives, such as stabilizing agents, preservatives, solubilizing agents, buffers and the like, as may be desired.

In certain embodiments, the present invention provides a pharmaceutical composition with a compound and a pharmaceutically acceptable carrier. The carrier may be a liquid formulation, and is preferably a buffered, isotonic, aqueous solution. Pharmaceutically acceptable carriers may also be excipients, such as diluents, carriers and the like, and additives, such as stabilizing agents, preservatives, solubilizing agents, buffers and the like, as hereafter described.

Formulation excipients may include but are not limited to polyvinylpyrrolidone, gelatin, hydroxy cellulose, acacia, polyethylene glycol, manniton, sodium chloride and sodium citrate. For injection or other liquid administration formulations, water containing at least one or more buffering constituents is preferred, and stabilizing agents, preservatives and solubilizing agents may also be employed. For solid administration formulations, any of a variety of thickening, filler, bulking and carrier additives may be employed, for example, starches, sugars, fatty acids and the like. For topical administration formulations, any of a variety of creams, ointments, gels, lotions and the like may be employed. For most pharmaceutical formulations, non-active ingredients may constitute the greater part, by weight or volume, of the preparation. For pharmaceutical formulations, it is also contemplated that any of a variety of measured-release, slow-release or time-release formulations and additives may be employed, so that the dosage may be formulated so as to effect delivery of a compound of the invention over a period of time. For example, gelatin, sodium carboxymethylcellulose and/or other cellulosic excipients may be included to provide time-release or slower-release formulations, especially for administration by subcutaneous and intramuscular injection.

Compounds described herein may be combined as the active ingredient in an admixture with a pharmaceutical carrier according to conventional pharmaceutical compounding techniques. The carrier may take a wide variety of forms depending on the form of preparation desired for administration, for example, oral, topical, parenteral (including intravenous), urethral, vaginal, nasal, dermal, transdermal, pulmonary, deep lung, inhalation, buccal, sublingual, or the like. In preparing compositions of the present invention for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating-agents, lubricants, binders, disintegrating agents and the like in the case of oral solid preparations such as, for example, powders, hard and soft capsules and tablets.

Tablets and capsules may represent an advantageous oral dosage unit form. If desired, a composition with a compound of the invention may be coated by standard aqueous or nonaqueous techniques. The amount of active compound is such that an effective dosage will be obtained. In another embodiment, sublingual pharmaceutical compositions may be employed, such as sheets, wafers, tablets or the like. The active compound may also be administered intranasally as, for example, by liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch or alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Compounds of the present invention may also be administered parenterally. Solutions or suspensions of active peptides may be prepared in water suitably mixed with a surfactant, such as hydroxypropylcellulose. Dispersions may also be prepared, such as dispersions in glycerol, liquid polyethylene glycols and mixtures thereof in oils. These preparations may optionally contain a preservative to prevent the growth of microorganisms. Lyophilized single unit formulations may also be utilized, which are reconstituted, such as with saline, immediately prior to administration.

Pharmaceutical forms suitable for injectable use may include but are not limited to, for example, sterile aqueous solutions or dispersions and sterile powders, such as lyophilized formulations, for the extemporaneous preparation of sterile injectable solutions or dispersions. The form may be sterile and must be fluid to the extent that it may be administered by syringe. The form must be stable under the conditions of manufacture and storage and may be preserved against the contaminating action of microorganisms such as bacteria and fungi. The carrier may be a solvent or dispersion medium containing, for example, water, ethanol, a polyol, for example glycerol, propylene glycol or liquid polyethylene glycol, suitable mixtures thereof, and vegetable oils.

If in an aqueous solution, compounds of the present invention may be appropriately buffered by means of saline, acetate, phosphate, citrate, acetate or other buffering agents, which may be at any physiologically acceptable pH, generally from about pH 4 to about pH 7. A combination of buffering agents may also be employed, such as phosphate buffered saline, a saline and acetate buffer, and the like. In the case of saline, a 0.9% saline solution may be employed. In the case of acetate, phosphate, citrate, acetate and the like, a 50 mM solution may be employed. In addition to buffering agents, a suitable preservative may be employed, to prevent or limit bacteria and other microbial growth. One such preservative that may be employed is 0.05% benzalkonium chloride.

Compounds of the present invention may be administered in a dried and particulate form. In a preferred embodiment, the particles are between about 0.5 and 6.0 μm, such that the particles have sufficient mass to settle on the lung surface, and not be exhaled, but are small enough that they are not deposited on surfaces of the air passages prior to reaching the lung. Any of a variety of different techniques may be used to make dry powder microparticles, including but not limited to micro-milling, spray drying and a quick freeze aerosol followed by lyophilization. With micro-particles, the compounds of the invention may be deposited to the deep lung, thereby providing quick and efficient absorption into the bloodstream. Further, with such approach penetration enhancers are not required, as is sometimes the case in transdermal, nasal or oral mucosal delivery routes. Any of a variety of inhalers may be employed, including but not limited to propellant-based aerosols, nebulizers, single dose dry powder inhalers and multidose dry powder inhalers. Common devices in current use include but are not limited to metered dose inhalers, which may be used to deliver medications for asthma treatment, chronic obstructive pulmonary disease and the like. Preferred devices include dry powder inhalers, designed to form a cloud or aerosol of fine powder with a particle size that is always less than about 6.0 μm.

Microparticle size, such as mean size distribution, may be controlled by means of the method of making. For micro-milling, the size of the milling head, speed of the rotor, time of processing and the like may control the microparticle size. For spray drying, the nozzle size, flow rate, dryer heat and the like may control the microparticle size. For making by means of quick freeze aerosol followed by lyophilization, the nozzle size, flow rate, concentration of aerosoled solution and the like may control the microparticle size. These parameters and others may be employed to control the microparticle size.

Compounds of the present invention may be therapeutically administered by means of an injection, typically a deep intramuscular injection, for example, in the gluteal or deltoid muscle, of a time release injectable formulation. In one embodiment, a compound may be formulated with a PEG, for example, poly(ethylene glycol) 3350, and optionally one or more additional excipients and preservatives, including but not limited to excipients such as salts, polysorbate 80, sodium hydroxide or hydrochloric acid to adjust pH, and the like. In another embodiment, a compound is formulated with a poly(ortho ester), which may be an auto-catalyzed poly(ortho ester) with any of a variable percentage of lactic acid in the polymeric backbone, and optionally one or more additional excipients. In one embodiment poly(D,L-lactide-co-glycolide) polymer (PLGA polymer) is employed, preferably a PLGA polymer with a hydrophilic end group, such as PLGA RG502H from Boehringer Ingelheim, Inc. (Ingelheim, Germany).

Formulations may be made, for example, by combining a compound in a suitable solvent, such as methanol, with a solution of PLGA in methylene chloride, and adding thereto a continuous phase solution of polyvinyl alcohol under suitable, mixing conditions in a reactor. In general, any of a number of injectable and biodegradable polymers, which may also be adhesive to polymers, may be employed in a time release injectable formulation. The teachings of U.S. Pat. Nos. 4,938,763, 6,432,438, and 6,673,767, and the biodegradable polymers and methods of formulation disclosed therein, are incorporated herein by reference. The formulation may be such that an injection is required on a weekly, monthly or other periodic basis, depending on the concentration and amount of compound, the biodegradation rate of the polymer, and other factors known to those of skill in the art.

### 5. Routes of Administration

Compounds and/or compositions of the present invention are suitable to be administered orally, topically, nasally, or parenterally. If administered by injection, the injection may be intravenous, subcutaneous, intramuscular, intraperitoneal or other means known in the art. Compounds may be formulated by any means known in the art, including but not limited to formulation as tablets, capsules, caplets, suspensions, powders, lyophilized preparations, suppositories, ocular drops, skin patches, oral soluble formulations, sprays, aerosols and the like, and may be mixed and formulated with buffers, binders, excipients, stabilizers, anti-oxidants and other agents known in the art. In general, any route of administration by which the compounds are introduced across an epidermal layer of cells may be employed. Administration means may thus include administration through mucous membranes, buccal administration, oral administration, dermal administration, inhalation administration, pulmonary administration, nasal administration, urethral administration, vaginal administration, and the like.

A compound of the invention may be administered by means of a time-release injectable formulation, such compound may be in a formulation with a PEG, poly(ortho ester) or PLGA polymer. In another aspect, a compound may be administered by means of an automated delivery device providing subcutaneous delivery, either continuous or intermittent. Any of the foregoing methods and formulations may be applicable for treatment of chronic conditions or syndromes, including but not limited to chronic congestive heart failure and particularly chronic decompensated congestive heart failure.

Compounds of the present invention may also be administered by transdermal administration, including by means of the delivery system, including the apparatus, but not limited to the methods as disclosed in U.S. Patent Application Publication 2006/0034903. Similarly, the hydrogel formulations and solid state formulations disclosed therein may be adapted for use with the compounds.

### 6. Dosage

### Therapeutically Effective Amount

The actual quantity of compounds administered to a patient will vary depending on the severity and type of indication, the mode of administration, the particular compound used, the formulation used, and the response desired.

The dosage for treatment is administration, by any of the foregoing means or any other means known in the art, of an amount sufficient to bring about the desired therapeutic effect. Thus, a therapeutically effective amount may be an amount of a compound or pharmaceutical composition that is sufficient to induce a desired effect, including but not limited to an anti-inflammation effect. Those of ordinary skill in the art will appreciate that a therapeutically effective amount may be administered by means of a single dose or multiple doses, and that compositions provided herein may contain a unit dose of a therapeutically effective amount.

In general, provided compounds are highly active. For example, a compound may be administered at about 10 μg/kg to about 50 mg/kg body weight, depending on the specific compound selected, the desired therapeutic response, the route of administration, the formulation and other factors known to those of skill in the art.

### 7. Combination Therapy

It is contemplated that a provided compound can be used in combination with other drugs or therapeutic agents.

In some embodiments, compounds as described herein are administered in combination with one or more other agents intended to treat the same condition, or disease. As used herein, additional therapeutic agents that are normally administered to treat a particular disease, or condition, are known as “appropriate for the disease, or condition, being treated.”

For example, in some embodiments, compounds of the present invention, or a pharmaceutically acceptable composition thereof, are administered in combination with other anti-inflammatory agents to treat inflammatory diseases and/or disorders. Examples of known anti-inflammatory agents include, but are not limited to, dexamethasone, indomethacin and clobetasol.

In some embodiments, compounds of the present invention are administered in combination with one or more other pharmaceutically active agents intended to treat a different disease, disorder, or condition. For example, in some embodiments, it may be desirable to administer an inventive compound in order to reduce inflammation while concurrently administering a different pharmaceutically active agent in order to achieve a different biological result.

To give but one example, it is known that transdermal administration of pharmaceutically active agents often causes skin irritation at the site of delivery. Indeed, it is not uncommon that a skin irritating agent (e.g., SDS) be administered prior to or concurrent with application of a transdermal device such as, for example, a transdermal patch, in order to facilitate the delivery. Applicant has found that addition or co-administration of a compound as described herein in combination with transdermal administration of another pharmaceutically active agent can reduce inflammation and/or irritation associated with the transdermal administration of the other pharmaceutically active agent.

Indeed, Applicant has found that addition or co-administration of AFC itself, or of other compounds that are structurally related to AFC (i.e., polyisoprenyl protein inhibitor compounds) can similarly reduce inflammation and/or irritation associated with the transdermal administration of another pharmaceutically active agent. Such polyisoprenyl protein inhibitor compounds useful in accordance with this aspect of the present invention include those disclosed in U.S. Patent Application Publication 2005/0277694, entitled “Topical compositions and methods for epithelial-related conditions” and/or those disclosed in J. S. Gordon, et al., “Topical N-acetyl-5-farnesyl-L-cysteine Inhibits Mouse Skin Inflammation, and Unlike Dexamethasone, its Effects Are Restricted to the Application Site.” J. Invest Dermatol. 2007. Yet other polyisoprenyl-protein inhibitor compounds that may be useful are disclosed in U.S. Pat. Nos. 5,043,268, 5,705,528 and 5,202,456. In certain embodiments, the polyisoprenyl-protein inhibitor compound is N-acetyl-5-farnesyl-L-cysteine (AFC). Amounts of the compound used in the device may vary, depending on many factors including the size of the device and its release characteristics, the amount of the pharmaceutical active agent and the estimated duration of action of the device. Broadly, amounts of the compound range from about 0.1% to about 10% w/v.

It is also known that single or chronic injections of a pharmaceutically active agent may sometimes result in inflammation, whether due to the identity of the pharmaceutically active agent (i.e., as an irritant) or to the mode of delivery. The present invention contemplates co-administration of one or more compounds of the present invention, and/or one or more other polyisoprenyl protein inhibitor compounds, in order to reduce inflammation associated with single or chronic injection of a pharmaceutically active agent. In these cases, the polyisoprenyl-protein inhibitor may be administered topically, formulated in an appropriate pharmaceutically acceptable carrier. See, e.g., U.S. Patent Application Publication Number 20050277694.

Exemplary pharmaceutically active agents whose delivery, whether transdermally or by injection, may cause skin irritation include levadopa, pro-drug forms of levadopa, insulin, estradiol, estrogen, progesterone, progestins, progestogen, testosterone, nicotine, nitroglycerin, cholinesterase inhibitors, stimulants, antidepressants, and analgesics.

To give another example, application of certain agents such as, for example, hair relaxants, which commonly are or contain basic agents (e.g., NaOH), can cause skin irritation (e.g., irritation and/or inflammation of the scalp). According to the present invention, one or more compounds of the present invention, and/or one or more other polyisoprenyl protein inhibitor compounds, can be administered together with such a hair relaxant (or other agent) to reduce skin irritation and/or inflammation.

Although the invention has been described in detail with particular reference to these preferred embodiments, other embodiments can achieve the same results. Variations and modifications of the present invention will be obvious to those skilled in the art and it is intended to cover all such modifications and equivalents. The entire disclosures of all references, applications, patents, and publications cited above and/or in the attachments, and of the corresponding application(s), are hereby incorporated by reference.

### EXAMPLES

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all classes, subclasses and species of each of these compounds, disclosed herein.

The following general experimental procedures were used in the Examples described below. Proton Nuclear Magnetic Resonance (¹HNMR) spectroscopy was recorded on a Bruker 500 MHz spectrometer, dimethyl sulfoxide (DMSO-d6), methanol (CD₃OD) or chloroform (CDCl₃) was used as ¹H-NMR solvent. The residual proton absorption of the deuterated solvent was used as the internal standard. All ¹H-NMR chemical shift are reported as 6 values in the parts per million (ppm). The splitting pattern abbreviations are as follows: s, singlet; d, doublet; t, triplet; q, quartet; br, broad; m, multiplet; dd, doublet of doublet; dt, doublet of triplets. The HPLC analysis was done using a phenomenex luna C₁₈(2)50×4.6 mm column. The mobile phase is 60% water, 40% acetonitrile containing 0.05% trifluoroacetic acid at 2 ml per minute flow rate for the first 2.5 minutes, followed by a gradient to 100% acetonitrile containing 0.05% TFA over 10 minutes. The eluent is observed at 214 nm.

### Example 1

### Synthesis of Tert-butyl 1-(hydroxyamino)-1-oxo-3-((2E,6E)-3,7,11-trimethyldodeca-2,6,10-trienylthio)propan-2-ylcarbamate (103)

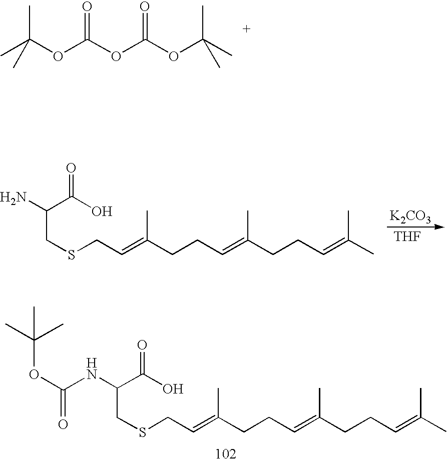

Farnesylcysteine (10 g, 30.7 mmol) was dissolved in THF (45 mL) and aqueous potassium carbonate (15.4 mL, 2M) with vigorous stirring and cooled to 5° C., then di-tert-butyl dicarbonate (13.4 g, 61.4 mmol) was added slowly as a solution in THF (15 mL) alternately with additional aqueous potassium carbonate (46.1 mL, 2M) in order to maintain the reaction mixture between about pH 9 and 10. When the addition was complete and the pH was stable, the reaction was allowed to warm to room temperature for three hours with continued stirring.

At the conclusion of the reaction period, the pH was adjusted to about 2 by addition of 1M aqueous HCl, and the product was extracted into ethyl acetate (3×100 mL). The combined organic phase was dried over anhydrous sodium sulfate, and the solvent was removed in vacuo. Crystals of tert-butanol were removed by gravity filtration using ethyl acetate to rinse. The solvent was again removed in vacuo to afford the product (13 g) as a yellow oil.

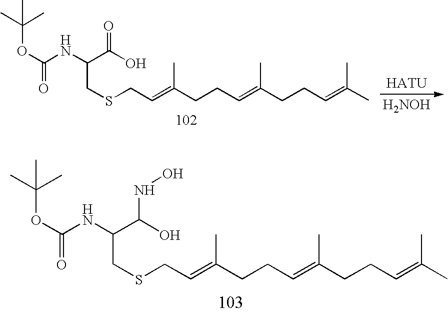

2-(tert-butoxycarbonylamino)-3-((2E,6E)-3,7,11-trimethyldodeca-2,6,10-trienylthio) propanoic acid (102) was reacted according to Scheme 1 with NH₂OH to yield the product 103, viscous yellow oil, 49% yield, HPLC (Phenomenex Luna Cl8 (2) 5 μm, 100 Å, 50 mm×4.6 mm, 2 ml/min, 2.5 min at 40% acetonitrile, 10 min grad. to 100% acetonitrile, observed at 214 nm): 8.45 min, >98% pure. Low res MS (API-ES Positive): 463.4 (M+Na). NMR: 1H(CDCl₃, 500 MHz): 1.33 (br s, 6H), 1.49 (br s, 9H), 1.56 (s, 6H), 1.82-2.01 (m, 8H), 2.71 (br s, 2H) 3.11 (br s, 2H), 4.18 (br s, 1H), 5.01 (s, 2H), 5.13 (s, 1H), 5.31 (br s, 1H).

### Example 2

### General Procedure for Synthesis of ACYL R³Substituted Compounds with Carboxylic Acid for R¹ on the Solid Phase

Farnesyl cysteine (FC) was protected with Boc as above (#102), then titrated to pH 7 with 2M aqueous cesium carbonate. The cesium salt was dried in vacuo, then rinsed with tetrahydrofuran (THF) and evaporated to dryness three times to drive off the remaining water. In the mean time, Merrifield resin was swelled in dichloromethane (DCM) for one hour, then drained and washed three times with N,N-dimethylformamide (DMF) and drained just prior to use. The dried FC cesium salt was resuspended in DMF and added to the resin in an Erlenmeyer flask, then covered with aluminum foil and placed in a shaking incubator at 60° C. and 250 RPM for 16 hours.

The next day, the reaction was removed from the incubator and washed successively with DMF three times, 1:1 DMF: Water three times, DCM three times, and methanol three times all in a fritted funnel under suction. After removal of the final methanol wash, the resin was dried under vacuum over potassium hydroxide pellets for 16 hours.

The amine was then deprotected by reaction with 50% trifluoroacetic acid (TFA) in DCM. This was added to the resin, which had been pre-swelled in DCM for one hour, and agitated on a rotary evaporator under normal atmospheric pressure for thirty minutes. The resin was then washed successively with tree portions of DCM, three portions of methanol, three additional portions of DCM, and finally three more portions of methanol, each of which was approximately five times the volume of swelled resin. After washing, the resin was again dried under vacuum over KOH.

At this stage, substituents at R⁴ may be introduced by reaction with R⁴—X where X is a leaving group such as a halogen. Acylation of the free amine is straightforward, and may be accomplished using either an acyl chloride, an anhydride (for simple R³), or a carboxylic acid activated with HATU. In all cases, about two equivalents of acyl donor relative to the resin loading (as determined by the weight difference after the reaction with FC) are used.

In the case of HATU coupling, the carboxylic acid was first stirred with HATU for 30 minutes. After this time the deprotected FC-derivatized resin, having been pre-swelled in DCM, rinsed with DMF, and drained under gravity, was added to the activated acid. The reaction conditions were as described before for the initial loading of the resin, as were the washing steps.

Depending on the reagents used above, the compound may be further derivatized, or cleaved from the resin. The cleavage reaction was carried out using 1M lithium hydroxide dissolved in 2:2:1 water:THF:methanol. The resin was washed with THF and drained, then the lithium hydroxide solution was added and the reaction mixture has shaken at 60° C. for 1 hour. The product was then filtered away from the resin, and the resin was washed with three portions of water and three portions of ethyl acetate, each of which was three times the volume of the resin. The filtrate was acidified to pH 3 and the product was extracted into ethyl acetate. The combined organic phases were dried over anhydrous sodium sulfate, and the solvent was removed in vacuo.

### Example 3

### Synthesis of Tetrazole Derivatives

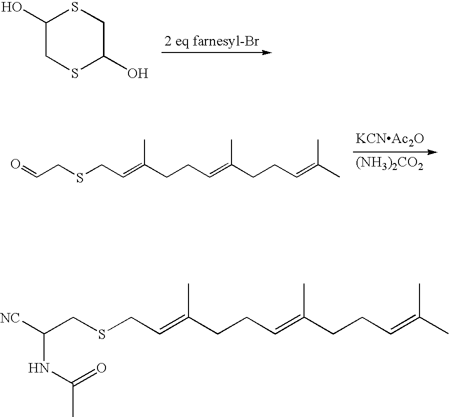

A nitrile intermediate may be synthesized by the reaction of 1,4-dithiane-2,5-diol with about two equivalents of an activated lipid, such as farnesyl bromide, under suitable conditions, such as in THF as solvent with K₂CO₃ as base at RT with stirring. The intermediate acetaldehyde may be converted to the nitrile by Strecker synthesis, for example by using KCN with (NH₃)₂CO₂ as base in water. This may be quenched with acetic anhydrides, or other anhydride as shown in Scheme 3, to add the desired R³ group and protect the free amine.

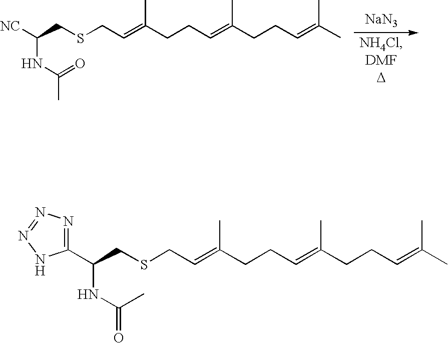

Following the methods described in Kumar et al. J. Org. Chem. 1996, 61:4462-4465, and Koguro, et al., Synthesis, 1998, 1998:910-914, heterocyclic derivatives at the R¹ position may be synthesized from a nitrile intermediate by heating with NaN₃ or a similar synthon in DMF and NH₄Cl as solvent.

### Example 4

### Synthesis of Substituted Amidine Derivatives

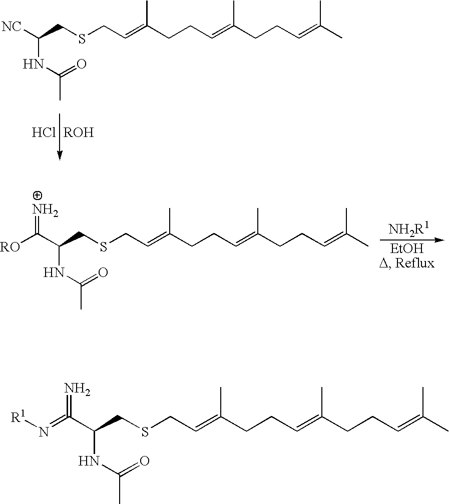

Following the methods described in Racanè, et al., Monatschefte für Chemie, 2006, 137:1571-1577 amidine derivatives at the R¹ position may be synthesized from a nitrile intermediate (see Example 3) by reacting first with an alcohol (ROH such as methanol) and acid and next by heating and refluxing with a snython with the formula NH₂R′ in EtOH as solvent, for example.

### Example 5

### Synthesis of Bis-Substituted Amidine Derivatives

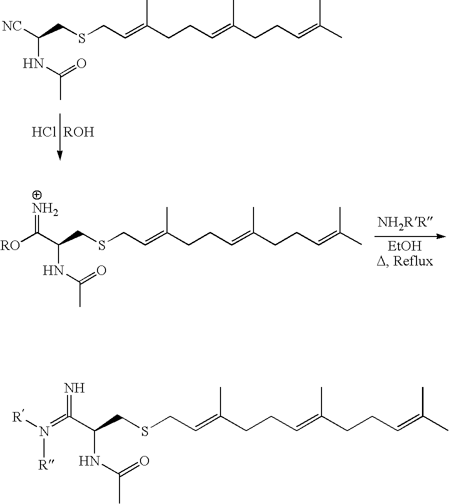

Following the methods described in Racanè, et al., Monatschefte für Chemie, 2006, 137:1571-1577 amidine derivatives at the R1 position may be synthesized from a nitrile intermediate (see example 3) by reacting first with an alcohol (ROH such as methanol) and acid and next by heating and refluxing with a snython with the formula NHR′R″ in EtOH as solvent, for example.

### Example 6

### Synthesis of Thioamide Derivatives

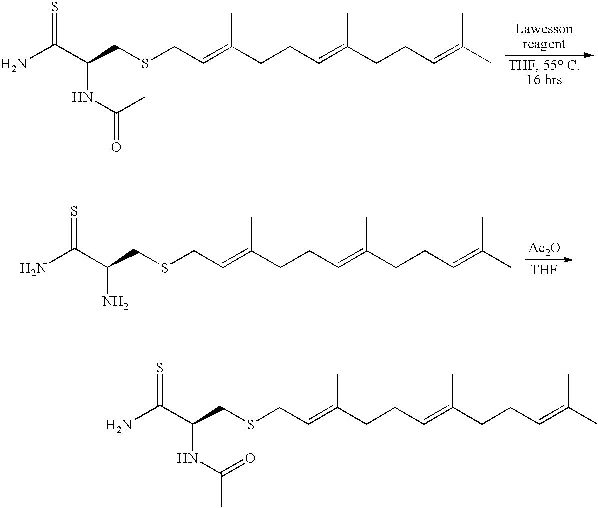

Methods of synthesis are described in M. P. Cava and M. I. Levinson, Tetrahedron, 1985, 41:5061-5087 and Z. Kaleta, et al, Org. Lett. 2006, 8:1625-1628. Starting from an amino-amide derivative of cysteine, the reaction may proceed first using Lawesson's reagent in THF as solvent. The second step involves modification of the free amine with acetic anhydride, a cyclic anhydride, or another synthon as needed to yield the desired R³, for example.

### Example 7

### General Synthesis of Sulfanomide Derivatives

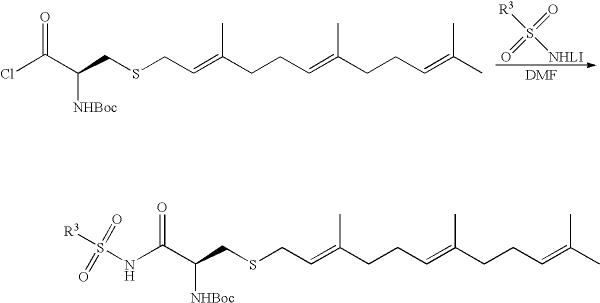

Methods of synthesis are described in Johansson et al. Journal of Combinatorial Chemistry 2000, 2:496-507; Rachita and Slough, Tetrahedron Letters, 1993, 34:6821-24; and Ishizuka, et al., Synthesis, 2000, 2000:784-88. Starting material may be a choloro-derivative of a boc-protected lipidated cysteine (such as #102 in example 1), or a choloroderavitive that is protected with another group (such as acetyl) on the amine, for example. This may be reacted with a substituted sulfonamide in DMF as solvent, for example.

### Example 8

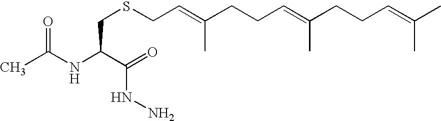

### Synthesis of Compound A:

In 50 mL round bottom flask, N-acetyl-S— farnesyl-L-cysteine (368 mg, 1.0 mmole) was dissolved in dichloromethane (10 mL). N-Cyclohexylcarbodiimide,N′-methyl polystyrene HL (1.7 g, 3.3 mole, from NovabioChem) was added followed by sufficient dichloromethane to swell the resin and keep the reaction mobile and the mixture gently magnetically stirred in 30 minutes. 5 mL of hydrazine in THF (1M) was added to the reaction solution and the reaction mixture was stirred in 3 hours at room temperature. The used resin was removed by filtration and washed with dichloromethane. Evaporation of the filtrate provided crude reaction mixture. The desired product was purified by preparative HPLC (305 mg, 80% yield): ¹H-NMR (500 MHz, CDCl₃) δ 1.60 (s, 6H), 1.67 (s, 6H), 1.70 (S, 3H), 1.92-2.09 (m, 8H), 2.75 (dd, J=7.9, 13.9 Hz, 1H), 2.88 (dd, J=5.7, 13.6 Hz, 1H), 3.20 (d, J=7.9 Hz, 2H), 4.51 (dd, J=7.6, 13.6 Hz, 1H), 5.10 (m, 2H), 5.24 (t, J=7.6 Hz, 1H); 13 C-NMR (125 MHz, CDCl₃) δ 16.0, 16.1, 16.2, 17.6, 17.7, 23.2, 23.4, 23.5, 25.7, 25.8, 26.2, 26.3, 26.5, 26.7, 30.0, 31.8, 31.9, 32.9, 33.2, 39.6, 39.7, 39.9, 51.3, 51.4, 119.6, 120.3, 123.6, 124.2, 131.4, 131.6, 135.5, 135.6, 135.8, 140.2, 140.3, 170.2, 171.1; ES-MS: mass calcd for Chemical Formula: C₂₀H₃₅N₃O₂S 381.2 (M+). Found (M+Na) m/z 404.3.

### Example 9

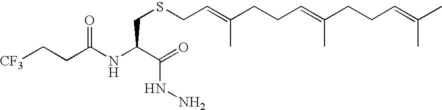

### Synthesis of Compound F:

In 50 mL round bottom flask, N-4,4,4-trifluorobutyl-5-farnesyl-L-cysteine (169 mg, 0.38 mmole) was dissolved in dichloromethane (10 mL). N-cyclohexylcarbodiimide,N′-methyl polystyrene HL (0.8 g, 1.5 mole, from NovabioChem) was added followed by sufficient dichloromethane to swell the resin and keep the reaction mobile and the mixture gently magnetically stirred in 30 minutes. 5 mL of hydrazine in THF (1M) was added to the reaction solution and the reaction mixture was stirred in 3 hours at room temperature. The used resin was removed by filtration and washed with dichloromethane. Evaporation of the filtrate provided crude reaction mixture. The desired product was purified by preparative HPLC (105 mg, 60% yield): ¹H-NMR (500 MHz, CD₃OD) δ 1.62 (s, 6H), 1.69 (s, 3H), 1.70 (S, 3H), 1.97-2.16 (m, 8H), 2.45-2.55 (m, 4H), 2.68 (dd, J=8, 14 Hz, 1H), 2.88 (dd, J=7.5, 13.5 Hz, 1H), 3.17-3.25 (m, 2H), 4.48 (t, J=7 Hz, 1H), 5.11 (m, 2H), 5.24 (t, J=6.5 Hz, 1H); ¹³C-NMR (125 MHz, CD₃OD) δ 16.1, 16.2, 17.8, 25.9, 27.4, 27.8, 29.0, 30.0, 30.2, 30.3, 30.5, 30.7, 33.7, 40.7, 40.9, 53.1, 121.4, 125.1, 125.5, 128.8 (q), 132.1, 136.3, 140.6, 171.9, 172.6; ES-MS: mass calc'd for Chemical Formula: C₂₂H₃₆F₃N₃O₂S 463.3 (M+). Found (M+Na) m/z 486.3.

### Example 10

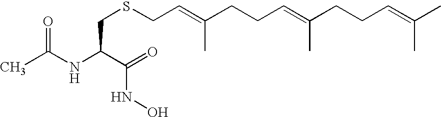

### Synthesis of Compound B:

In 50 mL round bottom flask, N-acetyl-5-farnesyl-L-cysteine (368 mg, 1.0 mmol) was dissolved in dichloromethane (10 mL). N-Cyclohexylcarbodiimide,N′-methyl polystyrene HL (1.7 g, 3.3 mole, NovabioChem) was added followed by sufficient dichloromethane to swell the resin and keep the reaction mobile and the mixture gently magnetically stirred in 30 minutes. Hydroxylamine (5 mL, 50% aqueous) was added to the reaction solution and the reaction mixture was stirred in 3 hours at room temperature. The reaction mixture was partitioned between water and ethyl acetate. The organic layer was separated and washed with aqueous bicarbonate solution (twice), then with water and finally with aqueous ammonium chloride. The organic layer was passed through silica gel plug and concentrated to yield the desired compound (172 mg) as viscous yellow oil in 45% yield. Low res MS (API-ES Positive): 406.4 (M+Na). NMR (¹H, CDCl₃, 500 MHz): 1.54 (br s, 6H), 1.62 (br s, 6H), 1.81-2.23 (m, 12H), 2.74-2.88 (m, 2H), 3.21 (br s, 2H), 4.52 (s, 1H), 5.12 (s, 2H), 5.24 (s, 1H), 10.5 (br s, 1H); NMR (¹³C, CDCl₃, 125 MHz): 13.1, 17.8, 23.0, 23.4, 25.7, 26.7, 29.9, 31.8, 32.0, 39.9, 45.9, 50.8, 119.4, 124.3, 124.5, 124.7, 131.7, 135.6, 140.2, 171.7, 173.3.

### Example 11

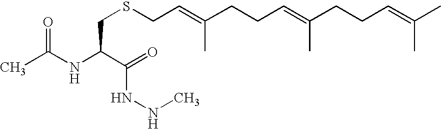

### Synthesis of Compound E:

In 100 mL round bottom flask, N-acetyl-5-farnesyl-L-cysteine (368 mg, 1.0 mmol) was dissolved in THF (20 mL). HATU (0.38 g, 1 mmol) was added, followed by addition of TEA (0.1 g, 1 mmol). The mixture was then magnetically stirred gently for 30 minutes. N-methyl hydrazine (0.1 g, 2.1 mmol) was added to the reaction solution. After stirring the reaction overnight, the reaction mixture was partitioned between water and ethyl acetate. The organic layer was separated and washed with aqueous bicarbonate solution (twice), then with water and finally with aqueous ammonium chloride. The organic layer was passed through silica gel plug and concentrated to yield the desired compound (257 mg, 65%). Low res MS (API-ES Positive): 418.3 (M+Na). NMR (¹H, CD₃OD, 500 MHz): 1.61 (br s, 6H), 1.69 (br s, 6H), 1.98-2.25 (m, 12H), 2.57-2.63 (m, 1H), 2.94-2.99 (m, 1H), 3.15 (s, 3H), 3.21 (br s, 2H), 5.08 (br s, 2H), 5.23 (br s, 1H), 5.62 (br s, 1H); NMR (¹³C, CD₃OD, 125 MHz): 15.2, 16.9, 19.3, 21.4, 22.8, 26.4, 28.0, 36.2, 37.6, 38.2, 38.6, 50.8, 119.5, 124.3, 124.6, 131.7, 135.2, 140.3, 172.4, 172.7.

### Example 12

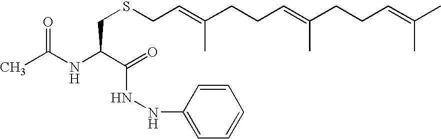

### Synthesis of Compound C:

In 100 mL round bottom flask, N-acetyl-5-farnesyl-L-cysteine (368 mg, 1.0 mmol) was dissolved in THF (20 mL). HATU (0.38 g, 1 mmol) was added, followed by addition of TEA (0.1 g, 1 mmol). The mixture was then magnetically stirred gently for 30 minutes. N-phenyl hydrazine (0.24 g, 2.1 mmol) was added to the reaction solution. After stirring the reaction overnight, the reaction mixture was partitioned between water and ethyl acetate. The organic layer was separated and washed with aqueous bicarbonate solution (twice), then with water and finally with aqueous ammonium chloride. The organic layer was passed through silica gel plug and concentrated to yield the desired compound (334 mg, 73%). Low res MS (API-ES Positive): 480.3 (M+Na). NMR (¹H, CD₃OD, 500 MHz): 1.59 (s, 6H), 1.62 (s, 6H), 1.91-2.08 (m, 12H), 2.72-2.77 (m, 1H), 2.82-2.93 (m, 1H), 3.21-3.35 (m, 2H), 5.09 (br s, 2H), 5.28 (br s, 1), 6.80 (t, 1H, J=7.8 Hz), 6.88 (d, 2H, J=7.8 Hz), 7.18 (t, 2H, J=7.8 Hz); NMR (¹³C, CD₃OD, 125 MHz): 16.2, 16.7, 21.4, 22.9, 24.4, 26.4, 28.02, 36.2, 37.6, 38.2, 38.9, 52.4, 117.4, 120.5, 124.3, 124.6, 124.7, 131.7, 135.2, 135.7, 140.3, 172.4, 172.5.

### Example 13

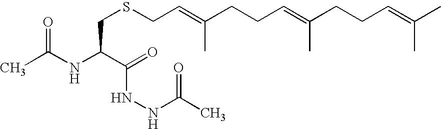

### Synthesis of Compound D:

In 100 mL round bottom flask, N-acetyl-5-farnesyl-L-cysteine (368 mg, 1.0 mmol) was dissolved in THF (20 mL). HATU (0.38 g, 1 mmol) was added, followed by addition of TEA (0.1 g, 1 mmol). The mixture was then magnetically stirred gently for 30 minutes. N-acetyl hydrazine (0.16 g, 2.1 mmol) was added to the reaction solution. After stirring reaction overnight the reaction mixture was partitioned between water and ethyl acetate. The organic layer was separated and washed with aqueous bicarbonate solution (twice), then with water and finally with aqueous ammonium chloride. The organic layer was passed through silica gel plug and concentrated to yield the desired compound (148 mg, 35%). Low res MS (API-ES Positive): 446.3 (M+Na). NMR (¹H, CD₃OD, 500 MHz): 1.54 (s, 6H), 1.65 (s, 6H), 1.92-2.12 (m, 12H), 2.72-2.74 (m, 1H), 2.87-2.97 (m, 1H), 3.21-3.35 (m, 2H), 5.09 (br s, 1H), 5.28 (br s, 1H); NMR (¹³C, CD₃OD, 125 MHz): 16.1, 17.0, 21.7, 23.1, 24.3, 26.2, 27.6, 30.5, 32.6, 33.7, 34.3, 40.9, 53.1, 121.1, 124.3, 125.9, 132.7, 136.6, 141.2, 172.2, 172.9, 174.3.

### Example 14

Table 3 below depicts % inhibition determined from an edema assay, an erythema assay, and myeloperoxidase (“MPO”) assay for compound A, compound B, compound C, compound D, compound E, and compound F.

TABLE 3

| Compound | | % Inhibition | | | |
|---|---|---|---|---|---|
| Number | Structure | Edema | MPO | Erythema | Dose |
| A | 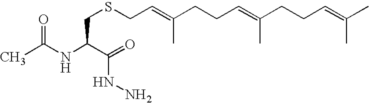 | — 42.36 ± 1.99 | — 93.53 ± 1.13 | — — | — 0.8 mg/20 μL |
| B | 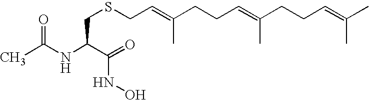 | — 33.64 ± 3.03 | — 87.45 ± 1.37 | — — | — 0.8 mg/20 μL |
| C | 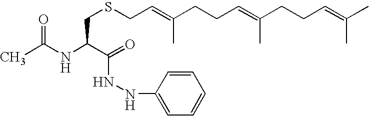 | — (−)0.34 ± 5.33 | — — | — 13.41 ± 11.92 | — 0.8 mg/20 μL |
| D | 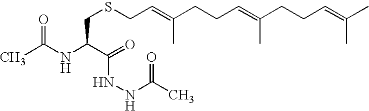 | — (−)10.5 ± 1.37 | — — | — (−)16.56 ± 29.9 | — 0.8 mg/20 μL |
| E | 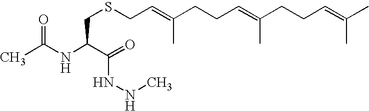 | — 5.85 ± 7.02 | — — | — 7.68 ± 8.15 | — 0.8 mg/20 μL |
| F | 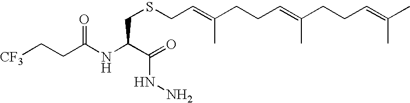 | 7.86 ± 0.98 — | 21.33 ± 12.68 — | 11.04 ± 7.28 — | 0.2 mg/20 μL — |

### Biological Examples

Described below are in vivo assays used to measure the biological activity of provided compounds, including the anti-inflammatory properties of the compounds, as measured by edema inhibition, erythema inhibition and MPO inhibition.

### Example 15

### Mouse Model of Inflammation-Edema, Erythema and MPO Background

The mouse ear model of contact irritation has been established as an appropriate model to determine whether topically applied anti-inflammatories inhibit the development of acute, chemically induced dermal irritation [see Van Arman, C. G. et al., Anti-inflammatory Drugs, Clin. Pharmacol. Ther. 16, 900-4 (1974); Young et al., Tachyphylaxis in 12-Otetradecanoylphorbolacetate-and Arachidonic Acid-Induced Ear Edema; J. Invest. Dermatol. 80:48-52, (1983); Tramposch et al., In Vivo Models of Inflammation, (Morgan D W, Marshall L A eds), Birkhaüser Verlag: Basel, pp 179-204, 1999; and Gordon et al., Topical N-Acetyl-S-Farnesyl-L-Cysteine Inhibits Mouse Skin Inflammation, and Unlike Dexamethasone, Its Effects Are Restricted to the Application Site, J. Invest. Dermatol., 128(3):643-54, 2008 March)]. Moreover, the mouse ear model has been used by various groups to identify and compare members of differing classes of anti-inflammatory agents with multiple mechanisms of action (reviewed in Tramposch et al., 1999, supra). The commonly used end points of inflammation are edema (Young et al., 1983, supra), (assayed by increase in ear thickness), neutrophil infiltration (which is measured by assaying for the neutrophil marker myeloperoxidase (“MPO”) (see Bradley et al., Cellular and Extracellular Myeloperoxidase in Pyogenic Inflammation, Blood, 60(3):618-22; 1982) and erythema (skin redness). Using this model, we investigated the in vivo anti-inflammatory activity of S-isoprenyl and S-farnesyl cysteine compounds to identify which structures possess physical or chemical properties critical for inhibiting innate inflammation in the skin.

### (a) Protocol-Edema Inhibition

The protocol for inducing in vivo acute contact inflammation on the ears of live mice has been described elsewhere (reviewed in Tramposch, 1999, supra). In brief, mice were sedated and their ears were treated with 1.2 μg/20 uL TPA (i.e., tetradecanoylphorbol-13-acetate). After 5 minutes, we dosed these TPA-treated ears with a single 8 μg/20 uL dose, a 2 ug/20 uL dose, or both doses, of the S-isoprenyl and S-farnesyl compounds. After 24 hours, the mice were sacrificed and edema was measured by taking micrometer readings of each ear. The percent inhibition of edema was determined by taking the average ear thickness of compound-treated ears and dividing it by the average thickness of 12 ears that only received TPA and subtracting that value from 100%. These values were corrected for the thickness of normal, non TPA-treated mouse ears of littermate controls. Results demonstrating percent inhibition of edema for representative compounds of the present invention are depicted in Table 3. ED₅₀ values were calculated as described in Gordon et al., “Topical N-acetyl-5-farnesyl-L-cysteine Inhibits Mouse Skin Inflammation, and Unlike Dexamethasone, its Effects Are Restricted to the Application Site”, J. Invest. Derm., Vol. 128 pp. 643-654 (2008).

### (b) Protocol-Erythema Inhibition

Another well documented biomarker of skin inflammation is skin redness, termed erythema, which is caused by capillary congestion and dilation in response to various chemical and environmental insults (see Denig, N. I. et al., Irritant Contact Dermatitis. Clues to Causes, Clinical Characteristics, and Control, Postgrad Med., May (1998); 103(5):199-200, 207-8, 212-3). The protocol for measuring erythema inhibition by S-isoprenyl and S-farnesyl cysteine compounds was developed in-house by utilizing the CR-400 chroma meter from Konica Minolta (http://www.konicaminolta.com/instruments/products/color/colorimeters/cr400-410/index.html). This instrument was used to measure the Δa* redness value from 6 mm biopsy punches taken 24 hours post TPA/compound treatment as described in the edema inhibition section above. The percent inhibition of erythema was determined by taking the average Δa* redness value of compound-treated ears and dividing it by the average Δa* value of 12 ears that only received TPA and subtracting that value from 100%. These values were corrected for the Δa* value of non TPA-treated mouse ears of littermate controls. Results demonstrating percent inhibition of erythema for representative compounds of the present invention are depicted in Table 3. Gordon et al., “Topical N-acetyl-5-farnesyl-L-cysteine Inhibits Mouse Skin Inflammation, and Unlike Dexamethasone, its Effects Are Restricted to the Application Site”, J. Invest. Derm., Vol. 128 pp. 643-654 (2008).

### (c) Protocol-MPO Inhibition

To assay for inhibition of dermal neutrophil infiltration by S-isoprenyl and S-farnesyl cysteine compounds, a standard method was used (see Bradley et al., 1982, supra; Young et al., 1983, supra; De Young et al, “Edema and Cell Infiltration in the Phorbol Ester-treated Mouse Ear are Temporally Separate and can be Differentially Modulated by Pharmacologic Agents”, Agents Actions, 26(3-4): 335-41 (March 1989); and Rao et al. (1993) Comparative Evaluation of Arachidonic Acid (AA)-and Tetradecanoylphorbol Acetate (TPA)-Induced Dermal Inflammation, Inflammation 17:723-41). Briefly, we homogenized 6 mm biopsy punches taken from both compound-treated ears as well as TPA-treated and non-treated control groups. We quantitated the levels of MPO by a calorimetric reaction that was measured spectrophotometrically. The percent inhibition of neutrophil infiltration by each S-isoprenyl and S-farnesyl cysteine compound was determined by comparing the average MPO levels in the presence and absence of these compounds. The calculation for percent inhibition of MPO was determined similar to that as described for calculating the percent edema inhibition, see the Edema Inhibition protocol, supra. Results demonstrating percent inhibition of MPO for representative compounds of the present invention are depicted in Table 3. Gordon et al., “Topical N-acetyl-5-farnesyl-L-cysteine Inhibits Mouse Skin Inflammation, and Unlike Dexamethasone, its Effects Are Restricted to the Application Site”, J. Invest. Derm., Vol. 128 pp. 643-654 (2008).

### EQUIVALENTS

Those skilled in the art will recognize, or be able to ascertain using no more than routine experimentation, that while the invention herein has been described with reference to particular embodiments, it is to be understood that these embodiments are merely illustrative of the principles and applications of the present invention and other embodiments may achieve the same results. It is therefore to be understood that numerous modifications may be made to the illustrative embodiments and that other arrangements may be devised without departing from the spirit and scope of the present invention as defined by the appended claims. The preceding examples may be repeated with similar success by substituting the generically or specifically described reactants and/or operating conditions used.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any targeting moiety, any disease, disorder, and/or condition, any linking agent, any method of administration, any therapeutic application, etc.) can be excluded from any one or more claims, for any reason, whether or not related to the existence of prior art.

Publications discussed above and throughout the text are provided solely for their disclosure prior to the filing date of the present application. Nothing herein is to be construed as an admission that the inventors are not entitled to antedate such disclosure by virtue of prior disclosure.

1. A compound of formula I: 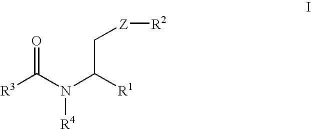
or a pharmaceutically acceptable salt thereof, wherein:
Z is —S—, —O—, —Se—, —S(O)—, —SO₂—, or —NH—;
is a heteroaryl group, or a moiety selected from 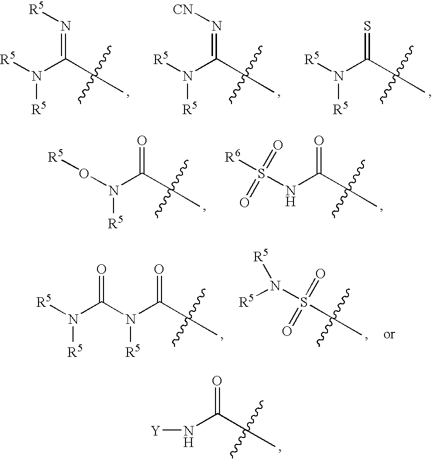
wherein at least one R⁵ group is H, and,
R⁵ is independently selected from H, alkyl, aryl, alkenyl, or alkynyl, wherein R⁵ is optionally substituted with one or two R⁷ groups;
R⁶ is H, alkyl, aryl, alkenyl, alkynyl, or a cyclic radical, where R⁶ is optionally substituted with one or two R⁷ groups;
Y is selected from H, —NH₂, —OH, —NH-phenyl, —NHC(O)CH₃, —NHCH₃, or —(C₁-C₈)alkyl;
R² is an aliphatic group substituted with one or more R⁷ groups;
R³ is alkoxy, aminoalkyl, alkyl, aryl, alkenyl, alkynyl, or a cyclic radical, where R³ is optionally substituted with one or two R⁷ groups;
R⁴ is H, alkyl, aryl, alkenyl, alkynyl, or a cyclic radical, where R⁴ is optionally substituted with one or two R⁷ groups; and
R⁷ is —NHC(═O)(C₁-C₈)alkyl, —(C₁-C₈)alkyl, —(C₁-C₈)alkenyl, —(C₁-C₈)alkynyl, phenyl, —(C₂-C₅)heteroaryl, —(C₁-C₆)heterocycloalkyl, —(C₃-C₇)cycloalkyl, —O—(C₁-C₈)alkyl, —O—(C₁-C₈)alkenyl, —O—(C₁-C₈)alkynyl, —O-phenyl, —CN, —OH, oxo, halo, —C(═O)OH, —COhalo, —OC(═O)halo, —CF₃, N₃, NO₂, —NH₂, —NH((C₁-C₈)alkyl), —N((C₁-C₈)alkyl)₂, —NH(phenyl), —N(phenyl)₂, —C(═O)NH₂, —C(═O)NH((C₁-C₈)alkyl), —C(═O)N((C₁-C₈)alkyl)₂, —C(═O)NH(phenyl), —C(═O)N(phenyl)₂, —OC(═O)NH₂, —NHOH, —NOH((C₁-C₈)alkyl), —NOH(phenyl), —OC(═O)NH((C₁-C₈)alkyl), —OC(═O)N((C₁-C₈)alkyl)₂, —OC(═O)NH(phenyl), —OC(═O)N(phenyl)₂, —CHO, —CO((C₁-C₈)alkyl), —CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈) alkyl), —OC(═O)O(phenyl), —S—(C₁-C₈)alkyl, —S—(C₁-C₈)alkenyl, —S—(C₁-C₈)alkynyl, and —S-phenyl, —SC(O)-phenyl, —SC(O)—(C₁-C₈)alkyl, —SC(O)—(C₁-C₈)alkenyl, —SC(O)—(C₁-C₈ alkynyl), —O—S(═O)₂—(C₁-C₈)alkyl, —O—S(═O)₂—(C₁-C₈)alkenyl, —O—S(═O)₂—(C₁-C₈)alkynyl, —O—S(═O)₂-phenyl, —(CH₂)ₙNH₂, —(CH₂)ₙ—NH((C₁-C₈)alkyl), —(CH₂)ₙN((C₁-C₈)alkyl)₂, —(CH₂)ₙNH(phenyl), or —(CH₂)ₙN(phenyl)₂, wherein n is 1 to 8.

2. The compound according to claim 1, wherein R¹ is a heteroaryl group.

3. The compound according to claim 2, wherein the heteroaryl group is selected from:

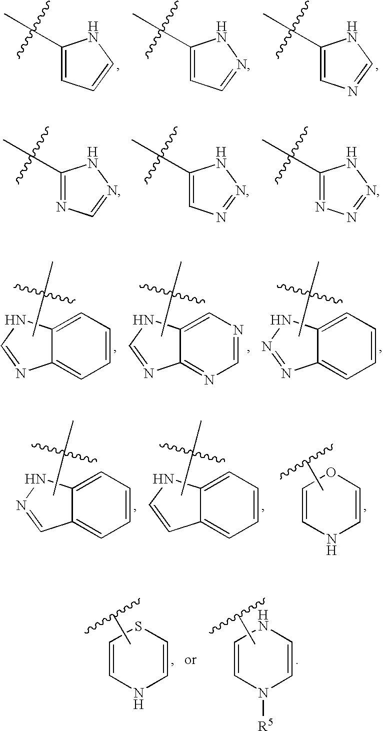

4. The compound according to claim 1, wherein R¹ is selected from:

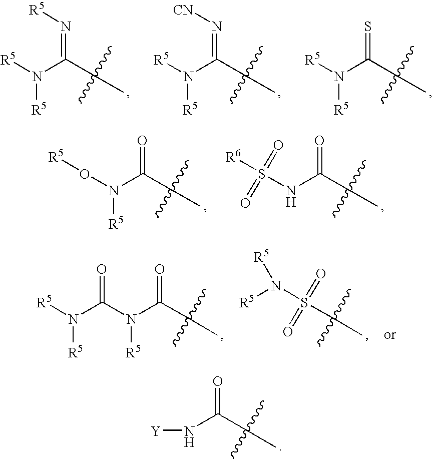

5. The compound according to claim 4, wherein R¹ is selected from: —C(O)NH—NH₂, —C(O)NH—OH, —C(O)NH—NH-phenyl, —C(O)NH—NHC(O)CH₃, or —C(O)NH—NHCH₃.

6. The compound according to claim 4, wherein Y is selected from —NH₂, —OH, —NH-phenyl, —NHC(O)CH₃, or —NHCH₃.

7. The compound according to claim 1, wherein R₂ is selected

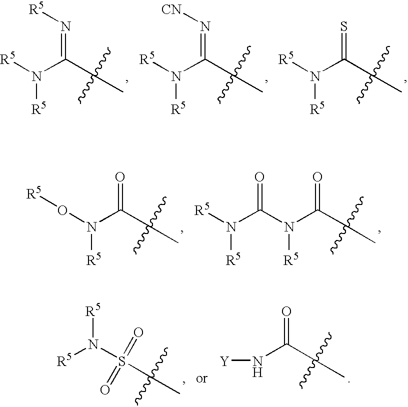

8. The compound according to claim 1, wherein R³ is —CH₃, —CH₂CH₃, —(CH₂)₂CH₃, —OC(CH₃)₃, phenyl, —(CH₂)₂C(O)OH, or —CH(CH₃)NHC(O)CH₃.

9. The compound according to claim 1, wherein R⁴ is H.

10. The compound according to claim 1, wherein R⁵ is H.

11. The compound according to claim 1, wherein R⁶ is H or —CH₃.

12. The compound according to claim 1, wherein Z is S.

13. The compound according to claim 1, wherein said compound is of formula Ia: 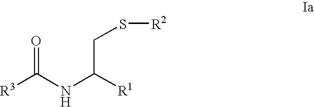
or a pharmaceutically acceptable salt thereof.

14. A compound selected from:

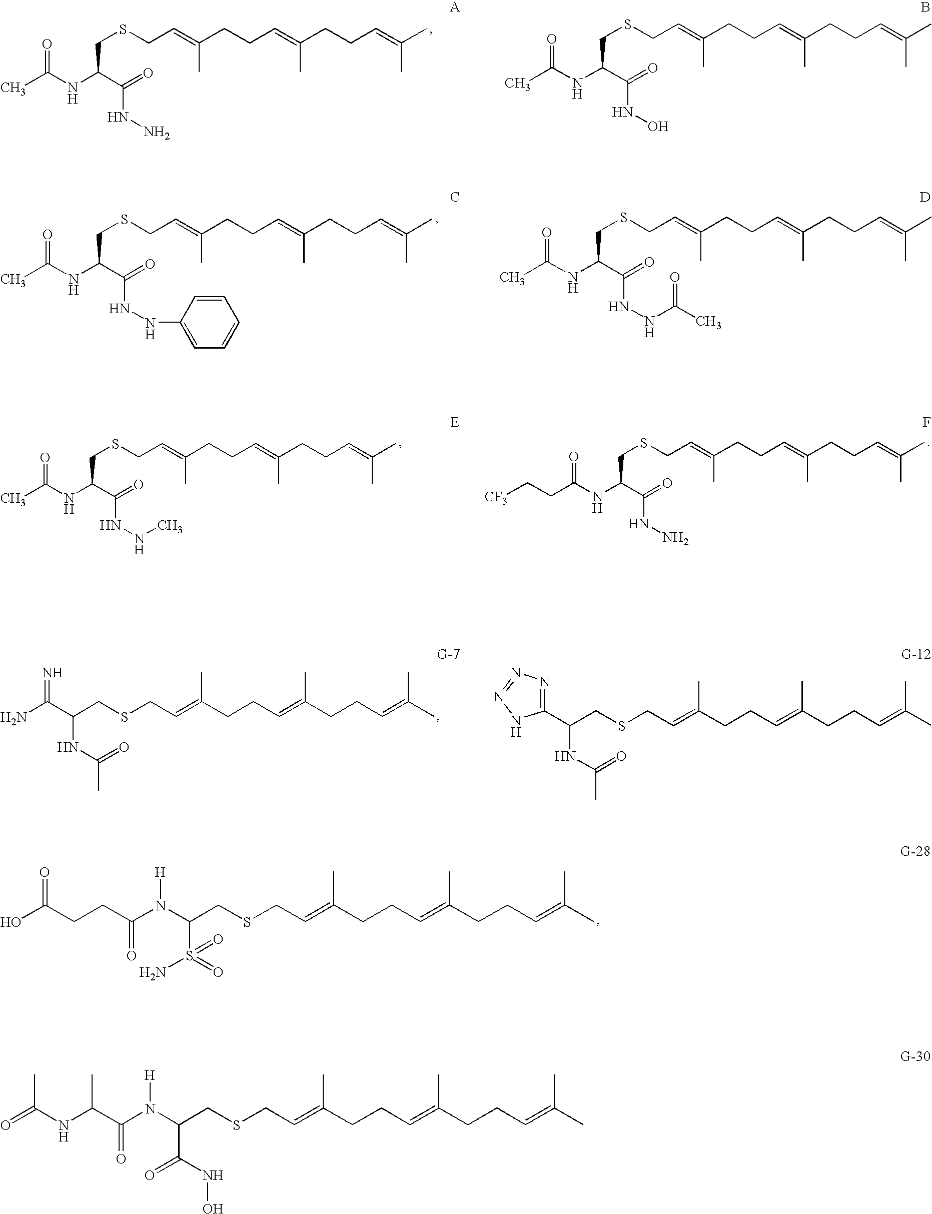

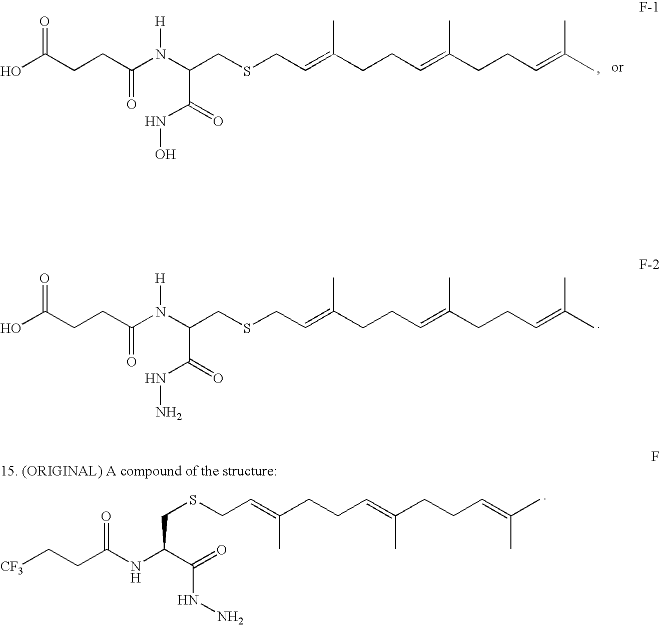

16. A composition comprising a compound according to claim 1, and a pharmaceutically acceptable adjuvant, carrier, or vehicle.

17. The composition according to claim 16, in combination with an additional therapeutic agent.

18. The composition according to claim 17, wherein the additional therapeutic agent is selected from the group consisting of dexamethasone, indomethacin and clobetasol.

19. A method for treating or lessening the severity of an inflammatory disease or disorder in a patient in need thereof, comprising the step of administering to said patient a compound according to Claim 1 or a composition thereof.

20. The method according to claim 19 wherein the disease or disorder is selected from inflammation, asthma, an autoimmune disease, COPD, inflammatory responses of the immune system, a skin disease, irritable bowel syndrome, and a neurodegenerative disorder, wherein the method comprises administering to a patient in need thereof a composition of the present invention.

21. The method according to claim 20 wherein the inflammation is acute or chronic.

22. The method according to claim 20 wherein the COPD is selected from emphysema, chronic bronchitis and a small airways disease.

23. The method according to claim 20 wherein the skin disease reduces acute skin irritation.

24. The method according to claim 20 wherein the skin disease is selected from rosacea, atopic dermatitis, seborrheic dermatitis, and psoriasis.

25. The method according to claim 20 wherein the irritable bowel syndrome is selected from Chron's disease and ulcerative colitis.

26. The method according to claim 20 wherein the central nervous system disorder is Parkinson's Disease.

27. The method according to claim 20, wherein the administering is achieved via transdermal delivery.

28. The method according to claim 27, wherein the administering is in combination with an additional therapeutic agent.

29. The method according to claim 28, wherein the additional therapeutic agent is selected from the group consisting of dexamethasone, indomethacin and clobetasol.

30. A method for treating or lessening the severity of an inflammatory disease or disorder in a patient in need thereof, comprising the step of co-administering transdermally to said patient a polyisoprenyl protein inhibitor compound and another pharmaceutically active agent.

31. The method according to claim 30, wherein the additional therapeutic agent is selected from the group consisting of dexamethasone, indomethacin and clobetasol.

32. The method according to claim 30, wherein the polyisoprenyl protein inhibitor compound is AFC.

33-39. (canceled)
